(19) United States
(12) Patent Application Publication
(10) Pub. No.: US 2026/0146069 A1
(43) Pub. Date: May 28, 2026

(54) RECOMBINANT OPCML FUSION PROTEIN OPTIMIZING OPCML D1 DOMAIN HOMODIMERISATION AND METHODS OF USE FOR CANCER TREATMENT

(71) Applicant: Papyrus Therapeutics, Inc., London (GB)

(72) Inventors: Hani GABRA, London (GB); Paul BLAKE, West Chester, PA (US)

(21) Appl. No.: 19/122,175

(22) PCT Filed: Oct. 26, 2023
(86) PCT No.: PCT/US2023/077953

§ 371 Date: Apr. 17, 2025

Related U.S. Application Data
(60) Provisional application No. 63/419,645, filed on Oct. 26, 2022.

(51) Int. Cl. C07K 14/47; A61K 9/00; A61K 38/00; A61P 35/00

(52) U.S. Cl. CPC C07K 14/4703; A61K 9/0019; A61P 35/00; A61K 38/00; C07K 2319/30

(57) ABSTRACT

Presented herein is a recombinant OPCML-Fc fusion protein molecule and methods of its use in cancer treatment where the Fc portion of the fusion protein molecule is only the CH2CH3 portion and lacks the hinge region containing the disulfide bridges preventing dimerization of the Fc portion and optimizing dimerization of the OPCML the D1 domain.

### CROSS REFERENCE

This application claims the benefit of U.S. Provisional Application No. 63/419,645 filed on Oct. 26, 2022, which is hereby incorporated by reference in its entirety for all purposes.

### INCORPORATION OF SEQUENCE LISTING

This application contains a Sequence Listing which has been submitted electronically in .XML format and is hereby incorporated by reference in its entirety. Said .XML copy, created on Oct. 26, 2023, is named “7071-0104PWO1.xml” and is 20,745 bytes in size. The sequence listing contained in this .XML file is part of the specification and is hereby incorporated by reference herein in its entirety.

### FIELD OF THE DISCLOSURE

The present disclosure relates to the field of medicine, more specifically to the field of cancer. In particular, to the role of OPCML in cancer prognosis, theragnosis, treatment, and research.

### BACKGROUND

Cancer is a leading cause of mortality worldwide, accounting for 7.9 million deaths (around 13% of all deaths) in 2007, with lung, stomach, liver, colon, and breast cancers causing the most deaths each year (World Health Organization; WHO). According to the WHO, deaths from cancer worldwide are projected to continue rising, with an estimated 12 million deaths in 2030. There is thus a very great need for additional and improved methods of treating cancers.

Opioid binding protein/cell adhesion molecule-like (OPCML) is a GPI anchored tumor suppressor protein that has been previously validated as an epigenetic biomarker for ovarian cancer. The OPCML gene shows frequent loss of heterozygosity in human tumors, is inactivated via extensive methylation of a CpG island in the promoter, and behaves as a tumor suppressor gene in in vivo models (WO 03/002,765 and Sellar et al. (2003) Nature Genetics 34 (3): 337-343). OPCML has also been found to be epigenetically inactivated and downregulated in a wider variety of cancers (Reed et al. (2007) Neuropathology and Applied Neurobiology 33(1): 77-85; Cui et al. (2008) PLOS ONE 3 (8): e2990).

OPCML acts at the external leaflet of the cell membrane at the lipid raft, interacting with and repressing a specific network of nine receptor tyrosine kinases (RTKs; Antony et al (2021) Cancer Gene Therapy 28(1-2): 18-26). A recombinant form of OPCML has been shown to be an effective treatment for HER-positive cancers, such as but not limited to HER2-positive ovarian, breast, renal, gastrointestinal, brain, lung, nasopharyngeal, endometrial, endometrial serous carcinoma, esophageal, gastric, colon, liver, cervical prostate, non-Hodgkin lymphoma, Hodgkin lymphoma, and nasal NK-T-cell lymphoma as well as for HER-negative cancers (Buza (2021) Arch Pathol Lab Med 145 (6): 687-691; WO/2011/128,701).

Still, improvements to the OPCML molecule are needed which would permit more efficient and effective administration with less pain or potential side effects for patients. Consequently, a need remains for alterations to the structure of the OPCML molecule so that it is able to reach its potential as a stand-alone effective cancer treatment.

### SUMMARY

We describe herein the design of two OPCML tumor suppressor biotherapeutic molecules in the form of an anchorless OPCML protein (r-OPCML PYTX-001) and in the form of a fusion protein joining a recombinant OPCML to a human IgG Fc peptide containing only the CH2CH3 region (rOPCML-Fc PYTX-004). The functional effects and mechanism of action of the two recombinant proteins were evaluated in vitro and rOPCML-Fc PYTX-004 evaluated intravenously in-vivo and shown to have a high degree of efficacy when used as a monotherapy treatment. In particular an intravenously administered rOPCML-Fc PYTX-004 therapeutic had good targeting affinity to human ovarian cancer patient derived xenograph (PDX) models. This molecule demonstrated substantial dose dependent efficacy in vitro and in vivo without compromising safety, making it an attractive biotherapeutic for movement into preclinical drug development.

### BRIEF DESCRIPTION OF THE DRAWINGS

FIG. 1 is a schematic of wild-type GPI anchored OPCML. A: shows the three IgG-like C2 domains (D1, D2, and D3) and the GPI-anchor attaching OPCML to a cell surface. B: provides the sequence for the wild-type human OPCML molecule (SEQ ID NO: 1). The sequences in bold font are the wild-type signal peptide (SEQ ID NO: 2) and the sequence for the region surrounding the wildtype GPI anchor site, which is underlined (SEQ ID NO: 8).

FIG. 2 shows the effects of Human serum albumin (HSA) on r-OPCML expression in HEK-293 cells. A: shows the sequences for three different signal peptides in bold font and the first 10 amino acids of human OPCML (underlined); wild-type human OPCML (SEQ ID NO: 3), ceruloplasmin (SEQ ID NO: 4), and HSA (SEQ ID NO: 5). B: western blot expression data and bar graph of western blotting densitometry for OPCML protein express associated with the wild-type OPCML, Ceruloplasmin, and HSA signal sequences.

FIG. 3 provides schematics of a new generation anchorless recombinant OPCML molecule (PYTX-001). A: is a schematic of the recombinant OPCML (rOPCML) molecule having the three OPCML IgG-like domains (D1, D2, and D3) where the wild-type GPI-anchor has been replaced with an alternative histidine tagged sequence. B: is a schematic of the now soluble recombinant OPCML (r-OPCML) molecule containing the histidine tag which can pass through HEK-293 cells into the cytoplasm. C: the sequence of the rOPCML molecule (SEQ ID NO: 6) where the wild-type OPCML signal sequence (MGVCGYLFLPWKCLVVVSLRLLFLVPT; SEQ ID NO: 2) has been replaced with the human serum albumin (HSA) sequence (shown in bold font; MKWVTFISLLFLFSSAYS; SEQ ID NO: 7) and where the wild-type OPCML sequence (TNKLGNTNASITLYGPGAVIDGVNSASRALAC LWLSGTLLAH FFIKF; SEQ ID NO: 8) surrounding and including the GPI anchor site (the underlined N being the GPI anchor site) has been replaced with TNKLGNTNASITLYGPGGGGLEVLFQGPRTGHHHHHHSSLEGPRFE (SEQ ID NO: 9). The His tag is shown in bold font. D: shows a schematic of the OPCML crystal structure consisting of three IgG-like domains that are homodimerized through domain 1.

FIG. 4 shows the sequence and structure of recombinant OPCML fused to a human IgG FC fragment containing only the CH2CH3 region (rOPCML-Fc). A: Sequence of rOPCML-Fc (SEQ ID NO: 10) where residues 1-18 are the human HSA signal peptide (bold font; SEQ ID NO: 7), underlined residues 19-28 are the initial part of the human OPCML protein, residues 29-109 are human OPCML Domain 1, residues 110-118 are the native human OPCML linker sequence, residues 119-202 are human OPCML Domain 2, residues 203-205 are the native human OPCML linker, residues 206-293 are human OPCML Domain 3, residues 294-298 are the native human OPCML linker, residues 299-301 are an artificial linker, residues 302-309 are the human rhinovirus 3C protease cleavage site (LEVLFQGP; SEQ ID NO: 11) used as a linker between OPCML and Fc, residues 310-315 are an artificial linker, residues 315-525 are human CH2CH3 IgG Fc fragment (residues 310-525 shown in italics; SEQ ID NO: 13), residues 526-528 are an artificial linker, and residues 529-534 are a cleavable polyhistidine tag (bold font). B: structural rendition of rOPCML-Fc PYTX-004 showing the three OPCML IgG-like domains that are homodimerized through domain 1 and the Fc portion of the molecule that is joined to OPCML domain 3.

FIG. 5 presents expression and purification data for PYTX-004. A: results of transfection with pcDNA3.1-rOPCML-Fc (pcDNA3.1-PYTX-004). B: subcloning results. C: results of serum withdrawal. D: results of affinity purification; Lane 1 is cell culture medium pre-binding; Lane 2 is effluent; Lane 3 is washing solution; Lane 4 is protein pre-stained marker; Lanes 5-10 are elution.

FIG. 6 shows that rOPCML-Fc impairs phosphorylation of receptor tyrosine kinases (RTKs) and inhibits downstream signaling in ovarian cancer cell lines. Cell culture media lacked 10% fetal calf scrum (FCS) and PYTX-001 protein or PYTX-004 protein (denoted by “−”) or contained 10% FCS. PYTX-001 protein or PYTX-004 protein (denoted by “+”). A: results for cell line SKOV3. B: results for cell line OVCAR8. Both the anchorless PYTX-001 and the CH2CH3 human IgG Fc-fused PYTX-004 molecules demonstrated equipotency in terms of on-target and downstream proximal pharmacodynamics, as well as equivalence in cancer functional assays.

FIG. 7 presents the expression and purification of rOPCML-Fc (PYTX-004). A: SDS-PAGE results: lane 1—standard marker; lane 2—rOPCML-Fc protein. B: HPLC results.

FIG. 8 shows that rOPCML-Fc PYTX-004 significantly inhibited proliferation of OVCAR3 and SKOV3 cells in vitro. A: IC50 measurements for cell lines OVCAR3, SKOV3, and PEO1. B: inhibition ability of rOPCML-Fc PYTX-004 on cancer cell line growth.

FIG. 9 shows that rOPCML-Fc PYTX-004 inhibited the wound healing of OVCAR3 and SKOV3 cells in vitro. A: OVCAR3 results. B: SKOV3 results. C: graphical illustration of OVCAR3 results. D: graphical illustration of SKOV3 results.

FIG. 10 shows that rOPCML-Fc (PYTX-004 construct) inhibited the invasion of OVCAR3 and SKOV3 cells in vitro. A: micrographs of treatment of OVCAR3 and SKOV3 cells with 0.05 or 0.1 mg/ml of OPCML-Fc PYTX-004 vs. control (NC). B: graphical illustration of treatment of OVCAR3 cells. C: graphical illustration of treatment of SKOV3 cells.

FIG. 11 shows that rOPCML-Fc (PYTX-004 construct) inhibited patient-derived organoids (PDO) of ovarian cancer in vitro. A: micrographs of treatment of OV016X-PDO. OV044-PDO, and OV041-PDO with 0.05 or 0.1 mg/ml of rOPCML-Fc vs. control (NC). B: graphical illustration of treatment of OV016X-PDO, OV044-PDO, and OV041-PDO.

FIG. 12 OPCML-Fc PYTX-004 protein downregulates pHER2, pAKT, and the RAF/MEK/ERK pathway in ovarian cancer cells. A: Cell culture media lacked EGF and rOPCML-Fc protein (denoted by “−”) or contained EGF or rOPCML protein (denoted by “+”). B: All cell culture media contained EGF (denoted by “+”) and contained no rOPCML-Fc protein (denoted by “−”) or some concentration of rOPCML-Fc protein.

FIG. 13 presents the approach used for developing a robust model of primary human ovarian tumors using patient derived xenografts (PDX) in NDG mice having severe T, B, and NK cell deficiencies.

FIG. 14 shows micrographic results of the PDX models, showing strong take rates.

FIG. 15 shows that OPCML-Fc PYTX-004 is active as a single agent applied intravenously in vivo. A: effect of rOPCML-Fc PYTX-004 on the growth of human ovarian cancer PDX in mice. B: tumor weight after treatment. C: tumor volume during treatment. D: animal body weight during treatment.

FIG. 16 shows that OPCML-Fc PYTX-004 targets tumor cells and inhibits proliferation. A: micrographs of OPCML-Fc PYTX-004 targeting tumor cells in the PDX model microenvironment using OPCML as a marker and showing enrichment primarily in tumor cells. B: inhibition of cancer cells with treatment of OPCML-Fc PYTX-004 using Ki-67 as a cancer cell marker.

### DETAILED DESCRIPTION

Unless defined otherwise, all technical and scientific terms used herein have the same meaning as commonly understood by one of ordinary skill in the art to which this disclosure belongs. Such terms are found defined and used in context in various standard references illustratively including J. Sambrook and D. W. Russell, Molecular Cloning: A Laboratory Manual, Cold Spring Harbor Laboratory Press; 4th Ed., 2012; F. M. Ausubel. Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; and Herdewijn, P. (Ed.), Oligonucleotide Synthesis: Methods and Applications, Methods in Molecular Biology. Humana Press, 2004.

Before the present disclosure is described in detail below, it is to be understood that this disclosure is not limited to the particular methodology, protocols and reagents described herein as these may vary. It is also to be understood that the terminology used herein is for the purpose of describing particular aspects only and is not intended to limit the scope of the present disclosure, which will be limited only by the appended claims.

Preferably, the terms used herein are consistent with the definitions provided in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland). Throughout this specification and the claims which follow, unless the context requires otherwise, the word “comprise”, and variations such as “comprises” and “comprising” will be understood to imply the inclusion of a stated member, integer, or step or group of members, integers, or steps but not the exclusion of any other member, integer, or step or group of members, integers, or steps.

As used in the disclosure and the appended claims. “and/or” refers to and encompasses any and all possible combinations of one or more of the listed items, as well as the lack of combinations when interpreted in the alternative (“or”). The term “about,” as used herein, encompasses the explicitly recited amounts as well as deviations therefrom of ±10%. For example, a deviation of 5% is encompassed by the term “about.” Several documents (for example: patents, patent applications, scientific publications, manufacturer's specifications, etc.) are cited throughout the text of this Specification. All of the documents cited herein are characterized as being “incorporated by reference.” In the event of a conflict between the definitions or teachings of such incorporated references and definitions or teachings recited in the present Specification, the text of the present Specification takes precedence.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.” For example, a condition A or B is satisfied by any one of the following: A is true (or present) and B is false (or not present), A is false (or not present) and B is true (or present), and both A and B are true (or present).

### Definitions

The term “affinity chromatography” as used herein refers to a specific mode of chromatography in which a ligand coupled to a stationary phase interacts with a molecule (e.g. an amino acid or portion of an immunoglobulin) in the mobile phase (the sample); i.e. the ligand has a specific binding affinity for the molecule to be purified. As understood in the context of the disclosure, affinity chromatography involves the addition of a sample containing a protein to a stationary phase which comprises a chromatography ligand.

The terms “affinity matrix” or “affinity separation matrix” or “affinity chromatography matrix”, as used interchangeably herein, refer to a matrix, e.g. a chromatographic matrix, onto which an affinity ligand (e.g., an Fc binding protein or a metal ion) is attached. The ligand (e.g., an Fc binding protein or metal ion) is capable of specific binding to at least a portion of a molecule of interest (e.g., a portion of an immunoglobulin or amino acid) which is to be purified or removed from a mixture.

The term “affinity purification” as used herein refers to a method of purifying a molecule of interest from a liquid or mixture by binding the molecule of interest to an appropriate ligand (e.g. an Fc binding protein or metal ion) that is immobilized to a matrix. Thereby, all other components of the liquid or mixture except the molecule of interest are removed. In a further step, the bound molecule of interest is eluted in purified form.

The term “amino acid sequence identity” as used herein refers to a quantitative comparison of the identity (or differences) of the amino acid sequences of two or more peptides or proteins.

The term “anion exchange chromatography” as used herein refers to removing from a sample one or more contaminants which bind to a positively charged resin. Examples of such contaminants include, but are not limited to, DNA, host cell proteins, endotoxins, and viruses. Bound molecules are eluted with an anion gradient.

The term “artificial” as used herein refers to an object that is not naturally occurring; i.e. the term refers to an object that has been produced or modified by man. For example, a polypeptide or polynucleotide sequence that has been generated by man (such as, for example, in a laboratory by genetic engineering, by shuffling methods, or by chemical reactions, etc.) or intentionally modified is artificial.

The term “binding” as used herein relates to the act or process by which one molecule attaches to another, selectively or stoichiometrically, by noncovalent forces. “Specific binding” means that at least a part of a molecule of interest binds stronger to a ligand for which it is specific compared to the binding to another non-specific target.

The term “binding activity” refers to the measured interaction of a molecule of interest to a ligand. For example, binding activity can be measured and determined for a molecule of interest, for example where a ligand is coupled to a matrix; that is, via an immobilized binding protein or metal ion.

The term “capture chromatography” as used herein refers to removing a molecule of interest from a sample. Examples of capture chromatography include, but are not limited to, the use of ligands such as protein A to capture Fc containing molecules or nickel to capture histidine tagged molecules. Oftentimes, capture chromatography relies on the capturing ligand being immobilized.

The term “chemotherapeutic agent” as used herein means any agent which has been approved for use as a chemotherapy for cancer. Examples include but are not limited to: all-trans retinoic acid, actimide, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, etoposide, fludarabine, fluorouracil, gemcitabine, hydroxyurea, idarubicin, irinotecan, lenalidomide, leucovorin, mechlorethamine, melphalan, mercaptopurine, methotrexate, mitomycin, mitoxantrone, oxaliplatin, paclitaxel, pemetrexed, revlimid, temozolomide, teniposide, thioguanine, thiotepa, valrubicin, vinblastine, vincristine, vindesine and vinorelbine. A chemotherapeutic agent for use in the combinations described herein may, itself, be a combination of different chemotherapeutic agents, suitable combinations include a combination of 5-fluorouracil (5-FU), leucovorin, and oxaliplatin (may be referred to as FOLFOX), or a combination of irinotecan, 5-FU, and leucovorin (may be referred to as IFL).

The term “chromatography” refers to separation technologies which employ a mobile phase and a stationary phase to separate one type of molecule from other molecules (e.g. contaminants) in the sample. The liquid mobile phase contains a mixture of molecules and transports these across or through a stationary phase (such as a solid matrix). Due to the differential interaction of the different molecules in the mobile phase with the stationary phase, molecules in the mobile phase can be separated.

The term “conjugate” as used herein relates to a molecule comprising or essentially consisting of at least a first protein attached chemically to other substances such as to a second protein or a non-proteinaceous moiety.

The term “deletion” or “amino acid deletion” as used herein refers to the removal of at least one amino acid at a particular position in a parent polypeptide sequence.

The term “disulfide bond,” or “disulfide bridge,” or “SS— bond” as used herein refers to a functional group present in some proteins and describes the covalent connection of two thiol groups, usually of cysteines, linking residues in either the same polypeptide chain or between two polypeptide chains.

The term “Fab” as used herein refers to the portion of an immunoglobulin molecule consisting of one intact light chain linked by a disulfide bond to the N-terminal part of the contiguous heave chain. Two Fab fragments are obtained from each IgG antibody molecule; each fragment contains one antigen-binding site.

The term “Fc binding protein” or “immunoglobulin-binding protein” or “Ig binding protein” is used to describe proteins that are capable of specifically binding to the Fc region of an immunoglobulin and/or to the Fc region of a fusion protein. Examples of Fc binding proteins include, without limitation, protein A, protein G, protein L protein Z, and others (see, for example, Choe et al. (2016) Materials 9:994).

“Fc binding proteins” or “Ig binding proteins,” as used herein, are capable of binding to entire immunoglobulins, and to immunoglobulin fragments comprising an Fc region, fusion proteins comprising at least an Fc region of an immunoglobulin, and conjugates comprising at least an Fc region of an immunoglobulin due to specific binding to the Fc region.

The term “Fc polypeptide.” “Fc fragment,” or “Fc region” as used herein refer to a crystallizable protein fragment of an immunoglobulin molecule. For example, that obtained from human IgG is a 50 kDa protein consisting of the C-terminal halves of two heavy chains linked by two disulfide bonds.

The term “fused” means that the components are linked by peptide bonds, either directly or via peptide linkers.

The term “fusion protein” relates to a protein comprising at least a first protein joined genetically to at least a second protein. A fusion protein can be created through joining two or more genes that originally coded for separate proteins. Thus, a fusion protein may comprise a multimer of identical or different proteins which are expressed as a single, linear polypeptide.

The term “hinge region” as used herein refers to a stretch of amino acids located between the Fab and Fc portions of an immunoglobulin molecule which contain a variable number of disulfide bonds: 2 for IgG₁ and IgG₄, 4 for IgG₂, and 11 for IgG₃.

As used herein, a subject is “in need of” a treatment if such subject would benefit biologically, medically or in quality of life from such treatment.

The term “insertions” or “amino acid insertion” refers to the addition of amino acids to the parent polypeptide sequence.

An “immunoglobulin” as used herein can include, but is not necessarily limited to, mammalian IgG, such as for example human IgG₁, human IgG₂, human IgG₄, mouse IgG, rat IgG, goat IgG, bovine IgG, guinea pig IgG, rabbit IgG; human IgM, human IgA; and immunoglobulin fragments comprising a Fc region.

The term “linker” as used herein refers in its broadest meaning to a molecule that covalently joins at least two other molecules. For example, a “linker” is a moiety that connects an Fc region with at least one further peptide, protein, or protein domain. In some aspects, the “linker” is a peptide linker, and can be one single amino acid or a peptide comprising two or more amino acids.

The term “modification” or “amino acid modification” refers to an exchange, a deletion, or an insertion of an amino acid at a particular position in a parent polypeptide sequence. Given the known genetic code, and recombinant and synthetic DNA techniques, the skilled scientist can readily construct DNAs encoding amino acid variants.

The term “operably linked” as used herein refers to a juxtaposition of two or more components (e.g., sequence elements) such that both components function normally and allow the possibility that at least one of the components can mediate a function that is exerted upon the at least one of the other components.

The term “parental” in the term “parental signal peptide,” “parental protein,” or “parental domain” as used herein refers to a region that is subsequently modified to generate a variant of said parental signal peptide, protein, or domain. Said parental signal peptide, protein, or domain may be a naturally occurring domain (for example, SEQ ID NO: 2), an artificial domain (for example, but not limited to, SEQ ID NO: 9), or a variant or engineered version of a naturally occurring domain (for example, SEQ ID NO: 13). The term “parental” is used interchangeably herein with the term “native” and in the same manner as described above for “parental.”

The terms “percent (%) amino acid sequence identity” or “percent identical” or “percent identity” with respect to a reference polypeptide sequence as used herein is defined as the percentage of amino acid residues in a sequence that are identical with the amino acid residues in the reference polypeptide sequence, after aligning the sequences and introducing gaps, if necessary, to achieve the maximum percent sequence identity.

To determine the sequence identity, the sequence of a query protein is aligned to the sequence of a reference protein. Methods for alignment are well-known in the art. For example, for determining the extent of an amino acid sequence identity of an arbitrary polypeptide relative to a reference amino acid sequence, the SIM Local similarity program is preferably employed (Xiaoquin Huang and Webb Miller (1991), Advances in Applied Mathematics, vol. 12:337-357), that is freely available (see also: http://www.expasy.org/tools/sim-prot.html). For multiple alignment analysis ClustalW is preferably used (Thompson et al. (1994) Nucleic Acids Res., 22 (22): 4673-4680). Preferably, the default parameters of the SIM Local similarity program or of ClustalW are used, when calculating sequence identity percentages.

In the context of the present disclosure, the extent of sequence identity is generally calculated with respect to the total length of the unmodified sequence, if not explicitly stated otherwise. Each amino acid of the query sequence that differs from the reference amino acid sequence at a given position is counted as one difference. The sum of differences is then related to the length of the reference sequence to yield a percentage of non-identity. The quantitative percentage of identity is calculated as 100 minus the percentage of non-identity.

The term “polishing chromatography” as used herein refers to additional chromatographic techniques that occur after at least one initial purification step and which are typically optimized to minimize levels of product aggregates such as HCP, DNA, and residual protein A. The chromatographic methods can include, without limitation, cation exchange, mixed mode (i.e, methods that use more than one form of interaction between the stationary phase and analytes to achieve separation) or hydrophobic interaction chromatography.

The terms “protein” and “polypeptide” refer to any linear molecular chain of two or more amino acids linked by peptide bonds and does not refer to a specific length of the product. Thus, “peptides”, “protein”, “amino acid chain,” or any other term used to refer to a chain of two or more amino acids, are included within the definition of “polypeptide.” and the term “polypeptide” may be used instead of, or interchangeably with any of these terms. The term “polypeptide” is also intended to refer to the products of post-translational modifications of the polypeptide, including without limitation, glycosylation, acetylation, phosphorylation, amidation, proteolytic cleavage, modification by non-naturally occurring amino acids and similar modifications which are well-known in the art. Thus, an Fc region comprising two or more protein domains also fall under the definition of the term “protein” or “polypeptide.”

The term “size exclusion chromatography” or “molecular sieve chromatography” as used herein refers to separation of molecules in a solution based on their size by filtration through a gel or matrix, such as spherical beads containing pores of a specific size distribution.

The terms “solid support” or “solid matrix” are used interchangeably herein for the stationary phase matrix in chromatography.

The term “subject” as used herein refers to an animal. Typically the animal is a mammal. A subject also refers to for example, primates (e.g., humans, male or female), cows, sheep, goats, horses, dogs, cats, rabbits, rats, mice, fish, birds and the like. In some aspects, the subject is a primate. In yet other aspects, the subject is a human.

The term “substitution” or “amino acid substitution” refers to an exchange of an amino acid at a particular position in a parent polypeptide sequence by another amino acid. For example, the substitution G46C refers to a Fc binding protein, in which the glycine at position 46 is replaced by a cysteine. For the preceding example, 46C refers to a cysteine at position 46. For the purposes herein, multiple substitutions are typically separated by a slash. For example, A1I/S11A/K35R/A46C refers to a variant comprising the combination of substitutions A1I, S11A, K35R, and A46C.

The term “therapeutically effective amount” refers to an amount of a substance that will elicit a biological or medical response of a subject, for example, reduction or inhibition of an enzyme or a protein activity, or ameliorate symptoms, alleviate conditions, or slow or delay disease progression, etc. In one non-limiting aspect, the term “therapeutically effective amount” refers to the amount of a protein disclosed that, when administered to a subject, is sufficient to achieve an immunomodulatory effect which at least partially alleviates, inhibits, prevents and/or ameliorates a cancerous condition.

The terms “treat”, “treating” or “treatment” of any disease or disorder as used herein refers in one aspect, to ameliorating the disease or disorder (i.e., slowing or arresting or reducing the development of the disease or at least one of the clinical symptoms thereof). In another aspect “treat”, “treating” or “treatment” refers to alleviating or ameliorating at least one physical parameter including those which may not be discernible by the patient. In yet another aspect, “treat”, “treating” or “treatment” refers to modulating the disease or disorder, either physically, (e.g., stabilization of a discernible symptom), physiologically, (e.g., stabilization of a physical parameter), or both. In yet another aspect, “treat”, “treating” or “treatment” refers to delaying the onset or development or progression of the disease or disorder.

The term “variant” as used herein includes an amino acid sequence that differs from another amino acid sequence by at least one amino acid substitution, deletion or insertion. These modifications may be generated by genetic engineering or by chemical synthesis or chemical reactions carried out by man. Variants can include, but are not limited to, a domain of a peptide, polypeptide, or protein, such as an Fc domain.

### DISCUSSION

OPCML is a member of the IgLON family, which consists of five cell-adhesion molecules: OPCML (aka OBCAM; IgLON1), NTM (aka HNT, NTRI, CEPU-1; IgLON2), LSAMP (aka LAMP; IgLON3), NEGR1 (aka KILON; MGC46680; Ntra; IgLON4) and IgLON5. They are members of the immunoglobulin (Ig) superfamily of cell adhesion molecules and are the most abundant glycosylphosphatidylinositiol-anchored protein expressed in neurons (Struyk et al. (1995) J Neurosci 15:2141-2156) and assist in neuronal growth and connections among nerve cells. The IgLON proteins contain three immunoglobulin (Ig)-like C2 domains Ig1, Ig2, and Ig3 (aka D1, D2, and D3, respectively) followed by a glycosylphophatidylinositiol anchor protein and possess 6 or 7 potential glycosylation sites (Itoh et al. (2008) Biochemistry 47:10132-10154). IgLON family members are found in many species, such as humans, apes, rodents, amphibians, fish, birds, and insects (Kubic et al. (2018). Evolutionary Bioinformatics Online 14:1-10 and identified references).

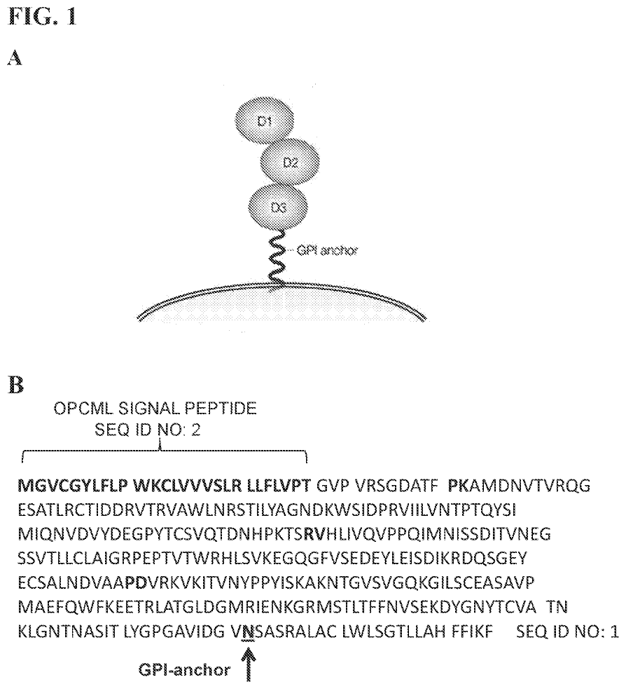

As noted above, and as shown in FIG. 1, the wild-type OPCML molecule consists of three IgG-like domains, typically referred to as domain 1 (D1), domain 2 (D2), and domain 3 (D3). The molecule has 345 amino acid residues, a signal peptide that directs the protein outside of the cell membrane, homodimerizes through D1, and is anchored to the external leaflet of the plasma membrane through a GPI anchor at asparagine 322 (FIG. 1A). With respect to post-translational modifications, N-linked glycosylation can occur at residues 44, 70, 140, 285, 293, and 306. Disulfide bonds occur between residues 57 and 115; 157 and 202; and 244 and 296. Lipidation signal occurs at residue 322, the GPI-anchor amidated asparagine.

Removing the GPI anchor region permits secretion of OPCML protein outside of the cell, into the external environment, where it can accumulate and be isolated. Previous studies had shown that replacing the native OPCML signal peptide with the Human serum albumin (HSA) signal peptide increased expression of OPCML (Kober et al. (2013) Biotechnology Bioengineering 110 (4): 1164-73). Consequently, replacing the native OPCML signal peptide with alternative signal peptides could increase the concentration of an anchorless OPCML molecule in the external environment.

Constructs were generated that expressed various recombinant OPCML molecules. Expression vectors are DNA molecules used to transfer and express foreign genetic material in a cell. Such vectors include a promoter sequence recognized by the host organism operably linked to the gene encoding the protein to be expressed. “Promoter” means a minimal DNA sequence sufficient to direct transcription of a DNA sequence to which it is operably linked. “Promoter” is also meant to encompass those promoter elements sufficient for promoter-dependent gene expression controllable for cell type specific expression; such elements may be located in the 5′ or 3′ regions of the native gene.

Expression vectors may also include a termination codon and expression enhancers. Any suitable vectors, promoters, enhancers and termination codons may be used to express the foreign genetic material from an expression vector. Suitable vectors include plasmids, binary vectors, phages, phagemids, viral vectors and artificial chromosomes (e.g. yeast artificial chromosomes or bacterial artificial chromosomes). Some expression vectors may additionally include one or more reporter genes encoding at least one reporter protein. An example of a reporter protein is a green fluorescent protein (GFP). Other expression vectors lack reporter genes.

There are various methods useful for integration of an exogenous nucleic acid molecule, such as the use of the CRISPR (Clustered Regulatory Interspaced Short Palindromic Repeats)/Cas9 method, TALEN (Transcription Activator-Like Effector Nuclease)-based method, or ZFN (zinc-finger nuclease)-based method.

A homology recombination (HR) construct for insertion of an exogenous nucleic acid molecule at a target site can be designed by those of skill in the art. The construct typically includes a first homology arm that is homologous to a sequence upstream of a target site and a second homology arm that is homologous to a sequence downstream of the target site. Each homology arm can include, for example, 200 to 1500 nucleotides (e.g., 200-250, 200-400, 250-500, 300-500, 400-600, 450-650, 500-800, 550-750, 650-900, 800-1000, 950-1200, or 1000-1500 nucleotides). The HR construct can further include multiple cloning sites between the two homology arms such that a gene to be inserted into the genome can be ligated into the construct. Alternatively, an HR construct containing the gene flanked by the two homologous sequences can be constructed using techniques known in the art. e.g., PCR. The HR construct can be used in a TALEN or CRISPR/Cas9 system to insert the nucleic acid molecule into the genome of the host cell.

TALEN and CRISPR/Cas9 methods both work by introducing a double-stranded DNA break in the genome at a target site. Based on the selected site, an HR construct harboring the nucleic acid molecule to be inserted at the target site can be designed and constructed.

CRISPR/Cas9 requires a gRNA specific to the targeted site and the endonuclease Cas9. The target site may be any sequence (about 20 nucleotides) that is unique compared to the rest of the genome and is immediately upstream of a Protospacer Adjacent Motif (PAM). Upon binding of the Cas9/gRNA complex to the target site. Cas9 cleaves the DNA. A skilled practitioner would be able to design a CRISPR/Cas9 construct directed at a target site.

TALEN utilizes a chimeric nuclease that contains an artificial DNA-binding domain of transcription activator-like effector (TALE) proteins and the catalytic domain of restriction endonuclease Fokl. As the code of DNA recognition by TALE proteins has been deciphered, an artificial DNA-binding domain for recognition of any DNA sequence can be designed. To minimize off-site effects. TALEN method can use a pair of chimeric nucleases that each recognizes a sequence on either side of the double-stranded DNA break site. A skilled practitioner would be able to design a TALEN construct directed at the selected site.

Zinc-finger nucleases (ZFNs) are artificial restriction enzymes generated by fusing a zinc finger DNA-binding domain to a DNA-cleavage domain. Zinc finger domains can be engineered to target specific desired DNA sequences, and this enables zinc-finger nucleases to target unique sequences within complex genomes. Each ZF set is linked to a cleavage domain, which must dimerize to cut DNA. Cleavage of the intended target gene can lead to disruption of its coding sequence by inaccurate repair through nonhomologous end joining. When a homologous donor DNA is introduced along with the ZFNs, it can be incorporated at the target by homologous recombination.

Molecular biology techniques suitable for the expression of polypeptides in cells are well known in the art. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

Suitable host cells for cloning or expressing the DNA in the vectors are prokaryotic, yeast, or other eukaryotic cells. Examples of useful mammalian host cell lines, without limitation, are mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/−DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK. ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2) and human embryonic kidney cells (HEK).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, signal peptide sequences, marker genes and other sequences as appropriate.

Alternatively, the sequence for a desired protein to be expressed by a construct can be sent to various organizations that generate the expression vector and confirm the protein sequence encoded therein. Examples of such organizations are Invitrogen Life Technologies (Carlsbad, CA), System Biosystems (Palo Alto, CA), PackGene (Zurich, Switzerland), to name but a few.

A recombinant OPCML (rOPCML) molecule was generated. To maximize secretion of the recombinant molecule, the native signal peptide and native linker existing before the OPCML protein was replaced with alternate signal peptides (i.e. ceruloplasmin and human serum albumin (HSA) signal peptides). However, any signal peptide capable of moving a protein through the plasma membrane and out of the cell can be used.

The sequence surrounding the GPI anchor site was also replaced with a sequence eliminating the GPI anchor and having a cleavable histidine tag. Use of the histidine tag permitted purification on a nickel column.

The construct was then transfected into a host cell according to methods well-established in the art. “Transfection” of a cell with a transgene of interest (typically meaning an expression cassette including the transgene) might result in transfected host cells (or transformed host cells, which is the same), wherein said host cells have integrated said transgene into their chromosomes. Said transgene might be integrated once or several times into said chromosome. That is, introducing the construct into the host cell leads to integration of the exogenous nucleotide sequence into the genome of the cell.

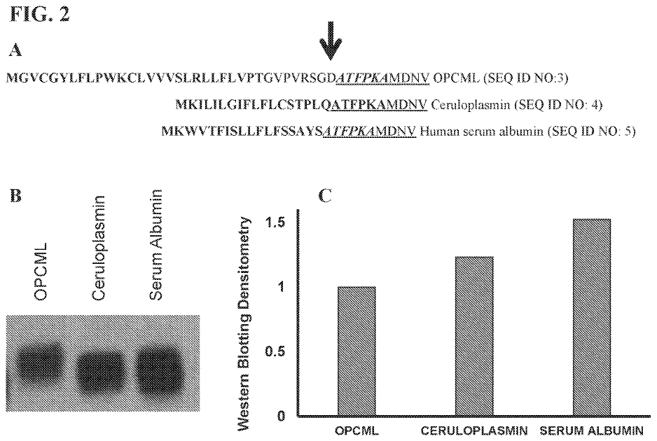

The constructs containing different signal sequences were tested in vitro in HEK-293 cells to identify the most efficient signal peptide (FIG. 2) and the HSA signal peptide chosen. While HEK-293 cells were used, any suitable mammalian cell line can be used such as, without limitation, those recited above or PER.C6. CAP™, HT-1080, NSO murine myeloma, SP2/0 murine hybridoma cells, C127 murine mammary gland cells, YB2/0 rat myeloma, etc.

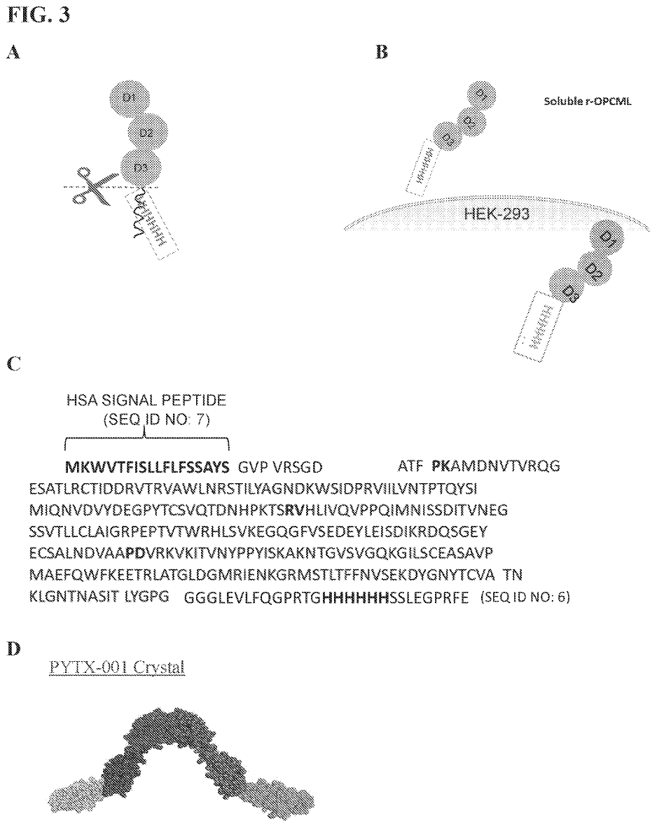

The recombinant HSA signal peptide construct generated, PYTX-001, had the sequence as shown in FIG. 3. This resulted in a recombinant OPCML protein molecule (rOPCML) that was soluble; that is, the anchorless rOPCML was capable of passing through the plasma membrane of HEK-293 cells into the culture medium and remained in solution.

Fusion to an IgG Fc region is an established strategy to extend the half-life of therapeutic proteins (Unverdorben et al. (2016) Mabs 8 (1): 120-128). Oftentimes the hinge region of human IgG, which is a stretch of heavy chains between the Fab and Fc portions of the molecule is included as well as the Fc portion (Rath et al. (2015) Crit Rev Biotechnol 35 (2): 235-254; Strohl (2015) Biodrugs 29 (4): 215-239). The hinge region is known to participate in dimerization via disulfide bonding at cysteine residues located in the hinge region. Since OPCML dimerizes via its D1 domain, to eliminate potential interference or destabilization due to the native OPCML dimerization and the hinge region dimerization, the hinge region was eliminated and only the CH2CH3 Fc portion used.

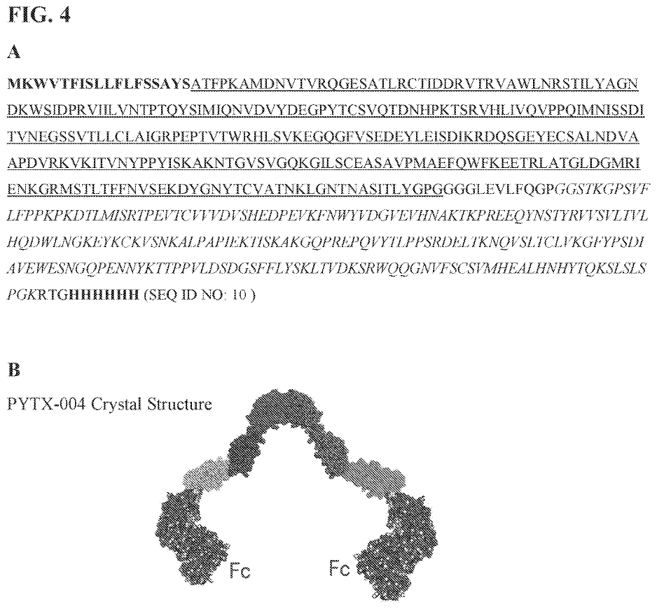

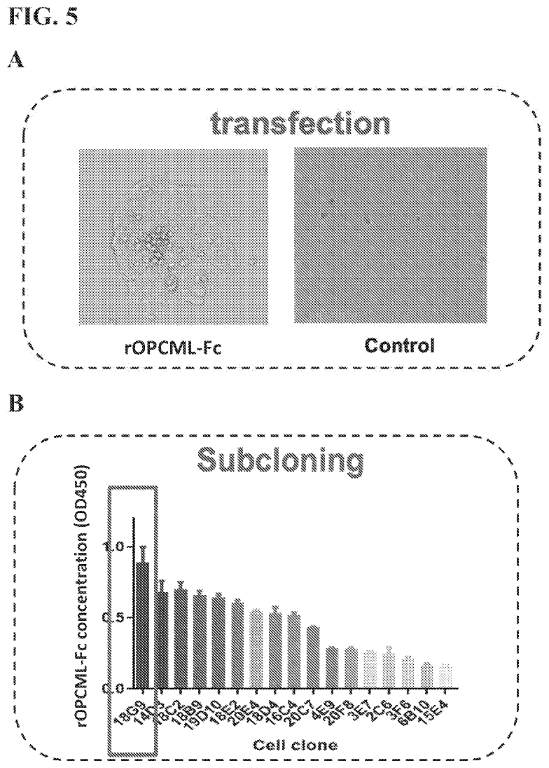

A new expression construct was created, PYTX-004, which contained the HSA signal peptide inserted 5′ to OPCML D1 and removal of the sequence surrounding the GPI anchor site. This GPI anchorless rOPCML molecule was then fused to an Fc molecule containing only the CH2CH3 region. FIG. 4 shows the sequence of the resulting rOPCML-Fc protein.

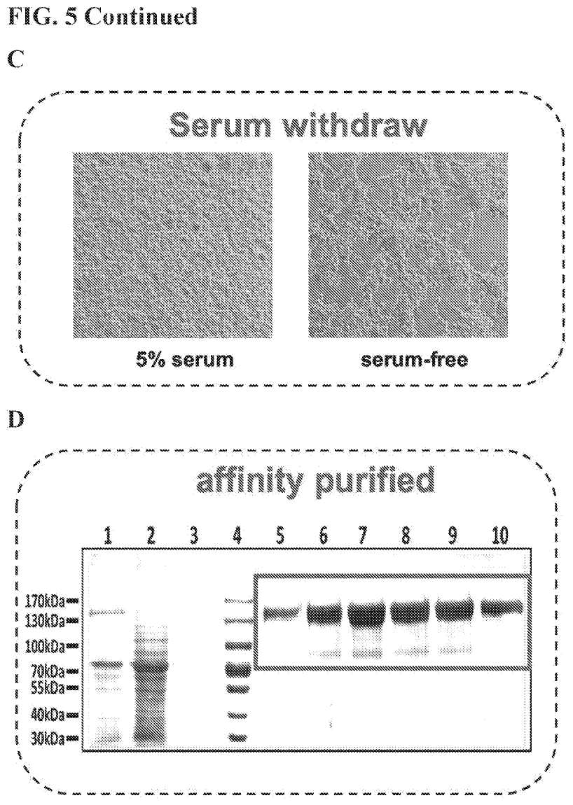

Stable transfection of the construct into appropriate cells was conducted according to methods well-established in the art. Appropriate cells include those listed above. Single cell cloning was then conducted to identify the highest producing single cell clones and one selected for further development and scale-up production (FIG. 5). To eliminate the potential for serum in the cell culture media to affect protein purification, the transfected cells underwent a serum concentration reduction scheme (FIG. 5C).

rOPCML-Fc protein from PYTX-004 was subsequently purified. Purification can be accomplished in any number of ways such as, without limitation affinity purification, affinity chromatography, anion exchange chromatography, capture chromatography, polishing chromatography, and/or size exclusion chromatography. Frequently, capture chromatography using protein A immobilized on a matirx is used to capture Fc containing molecules or nickel, cobalt, or copper immobilized on a matrix is used to capture His-tagged molecules. The rOPCML-Fc PYTX-004 protein was purified to high purity (FIG. 5D and FIG. 7), was then characterized.

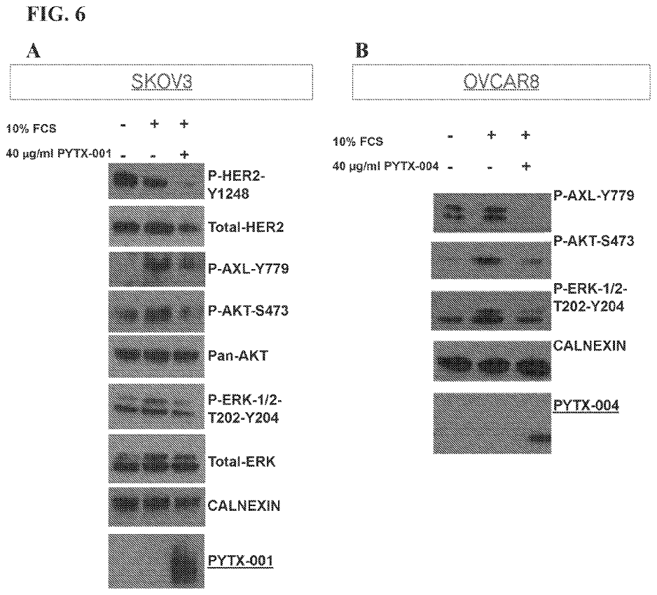

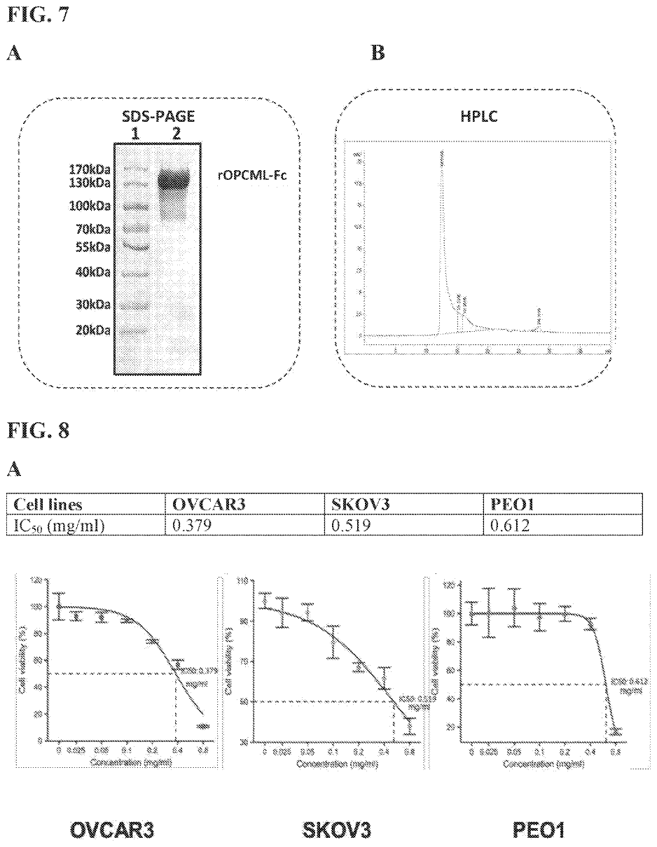

To determine whether the fused CH2CH3 Fc region influenced the signaling activity of rOPCML-Fc, its activity was compared to that of the rOPCML protein (PYTX-001 construct). Here ovarian cancer cell lines were treated with fetal calf serum (FCS) and purified rOPCML or rOPCML-Fc protein. FIG. 6 shows the results, indicating the equipotency of on-target and downstream proximal pharmacodynamics.

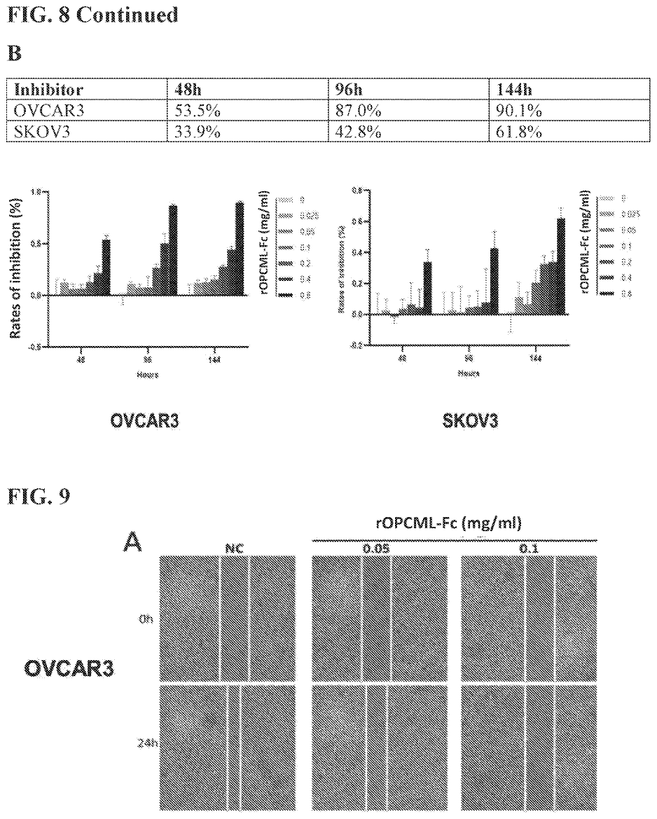

The ability of rOPCML-Fc protein to evoke significant signaling was further explored along with its effect on ovarian cancer cells. Three different cell lines were selected, OVCAR3, SKOV3, and PEO1, and the IC50 of rOPCML-Fc determined for each. As can be seen from FIG. 8A, rOPCML-Fc had an IC50 of 0.379 mg/ml for OVCAR3, of 0.519 mg/ml for SKOV3, and of 0.612 mg/ml for PEO1. The ability of different concentrations of rOPCML-Fc to inhibit OVCAR3 and SKOV3 cell growth was also determined for time periods of 48, 96, and 144 hours (FIG. 8B).

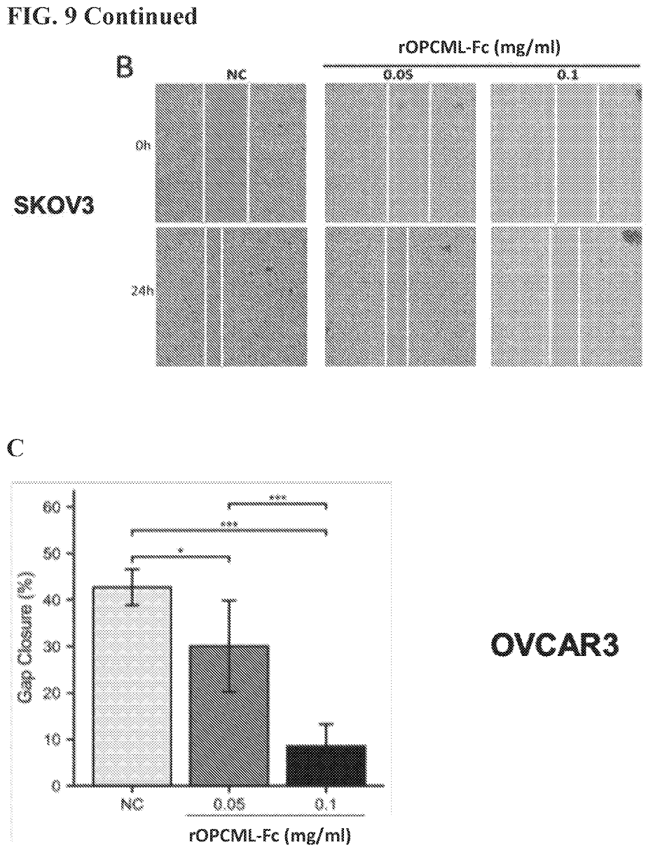

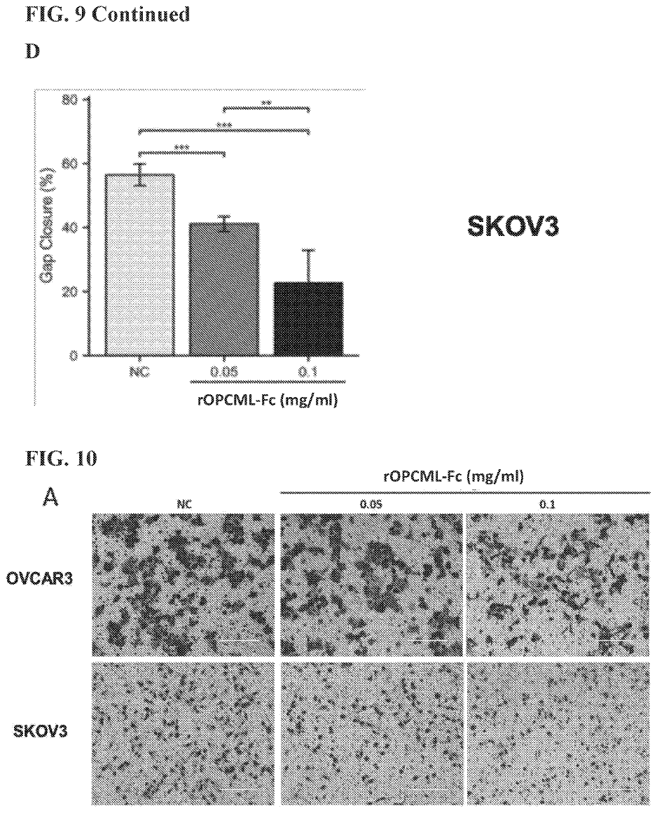

The effect of rOPCML-Fc on wound healing was also determined for OVCAR3 and SKOV3 cells in vitro using a gap closure assay (Birtley et al. (2019) Nature Communications 10:3134). As can be seen in FIGS. 9A and 9B, administration of 0.05 or 0.1 mg/ml rOPCML-Fc inhibited the ability of OVCAR3 and SKOV3 cells to infiltrate and close a gap within 24 hours of rOPCML-Fc administration. FIGS. 9C and 9D show this data in graph format and indicate that while both concentrations had positive inhibitory effects, the difference between the control and administration of 0.1 mg/ml rOPCML-Fc was highly significant for both cell types.

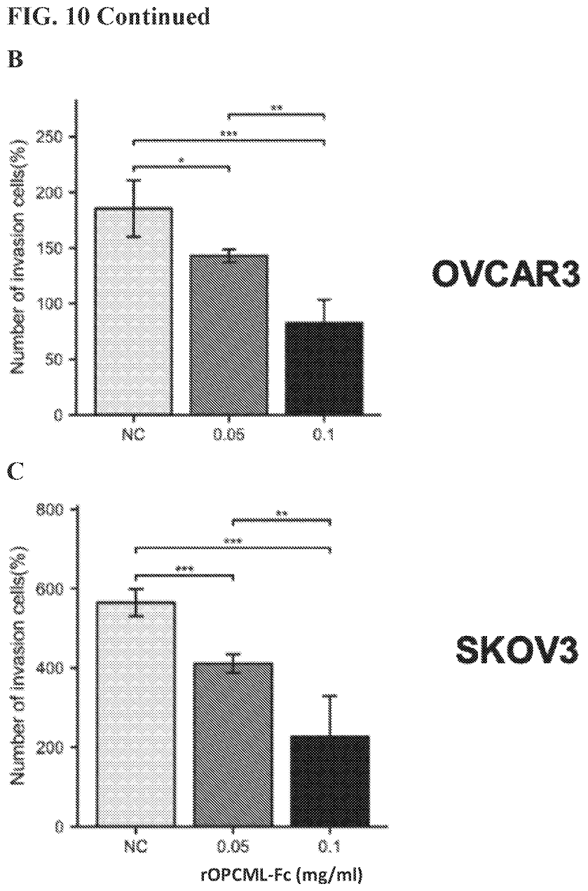

Similarly, the ability of rOPCML-Fc to inhibit the invasion of OVCAR3 and SKOV3 cells was also determined. Using the Birtley et al. invasion assay (Birtley et al. (2019) Nature Communications 10:3134), either 0.05 or 0.1 mg/ml rOPCML-Fc was applied. FIG. 10A shows the micrographic results while FIGS. 10B and 10C show the data in graph format for OVCAR3 and SKOV3, respectively. Again, while both concentrations had positive inhibitory effects, the difference between the control and administration of 0.1 mg/ml rOPCML-Fc was highly significant for both cell types.

Taken together, the results of FIGS. 8, 9, and 10 indicate that rOPCML-Fc powerfully inhibits invasion and migration in OVCAR3 and SKOV3 cells.

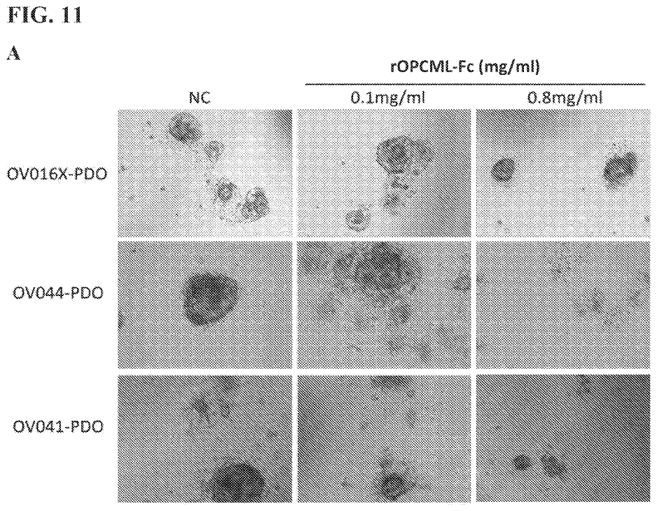

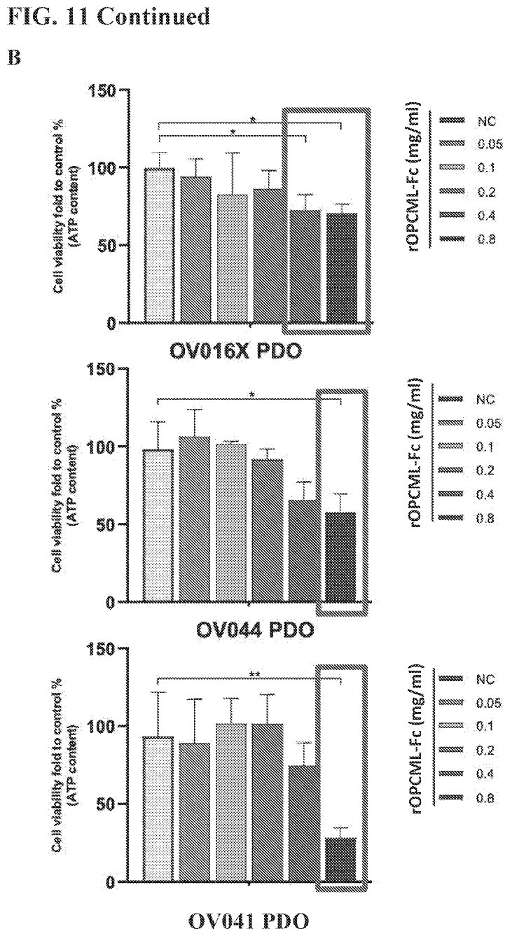

The effect of rOPCML-Fc protein on ovarian cancer patient-derived organoids (PDO) was explored. Here, organoids were used that were derived from ovarian tumor tissues obtained during the initial operation of patients diagnosed as serous carcinoma of the ovary. Cells from the organoid tissues were treated with 0.1 mg/ml or 0.8 mg/ml rOPCML-Fc. Results are presented in FIG. 11A. Cells were also treated with rOPCML-Fc at concentrations of 0 mg/ml, 0.05 mg/ml, 0.1 mg/ml, 0.2 mg/ml. 0.4 mg/ml, or 0.8 mg/ml. Results are shown in FIG. 11B. A statistically significant decrease in cell viability/proliferation activity was seen for treatment with 0.4 mg/ml or 0.8 mg/ml rOPCML-Fc in OV016X PDO and with 0.8 mg/ml rOPCML-Fc in OV044 PDO. A highly statistically significant decrease in viability/proliferation activity was seen for administration of 0.8 mg/ml rOPCML-Fc in OV041 PDO.

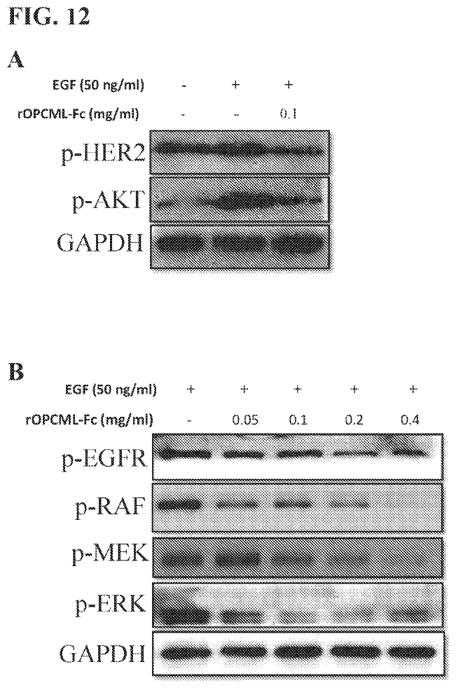

Additional signaling tests were conducted using cells stimulated with EGF. Here, rOPCML-Fc downregulated pHER 2 and the RAF/MEK.ERK pathway in ovarian cancer cells (FIG. 12).

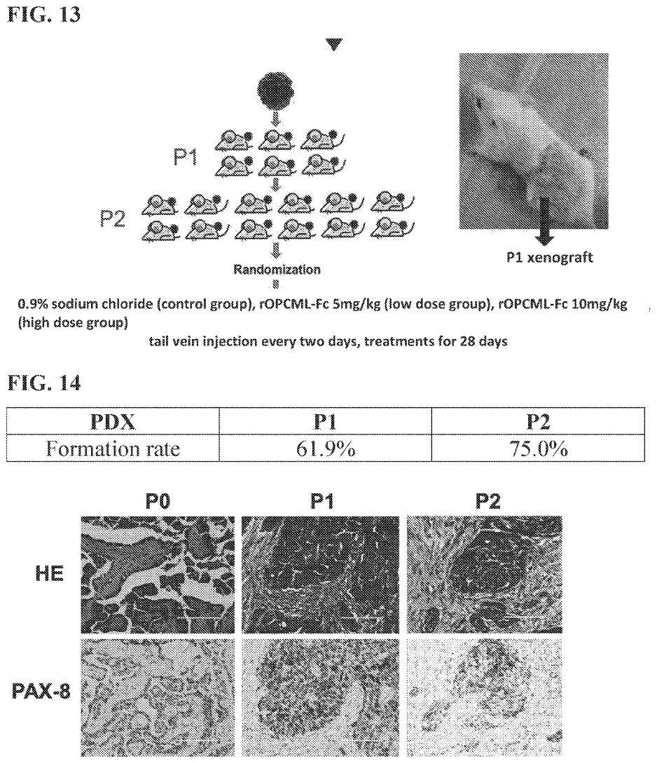

To determine the effects and safety of intravenous application of rOPCML-Fc, a robust model of primary human ovarian tumors, summarized in FIG. 13, was developed. Here, primary human ovarian tumor cells were introduced into NDG mice that had severe T, B, and NK cell deficiencies to create a patient-derived xenograft (PDX) model. FIG. 14 shows results of ovarian cancer marker PAX-8 for P0, the P1 generation, and the P2 generation, indicating that there was a strong uptake rate for even the P1 generation (61.9%) as well as the P2 generation (75.0%), creating a robust PDX model.

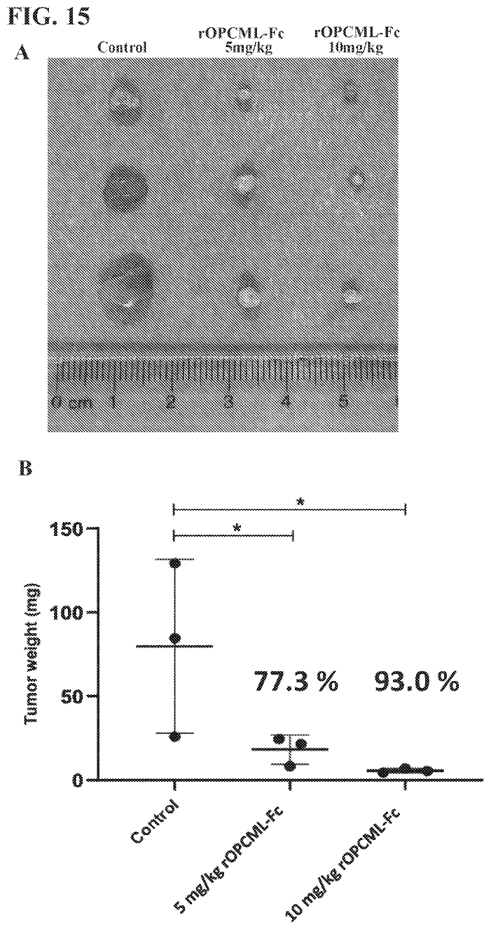

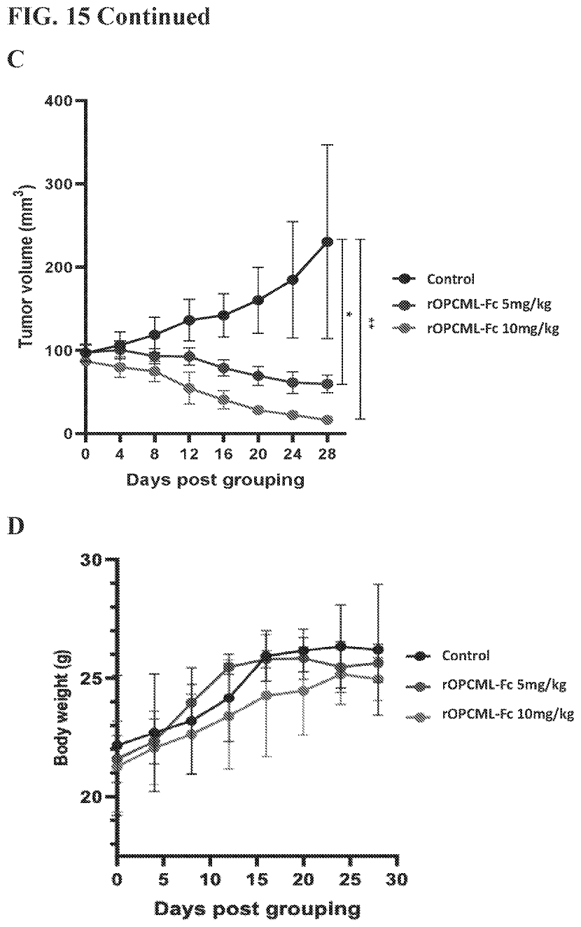

Using this model, two experimental groups were generated for rOPCML-Fc administration: a low dose group (5 mg/kg) and a high dose group (10 mg/ml) of rOPCML-Fc. Intravenous injection into the tail vein was administered every two days for 28 days. FIG. 15A-C shows that administration of only rOPCML-Fc was sufficient to inhibit the human ovarian PDX in a dose dependent fashion at both the 5 mg/kg and 10 mg/kg levels, reducing tumor weight by 77.3% and 93.0%, respectively. Importantly, intravenous administration of rOPCML-Fc protein did not affect the daily activities and weight of the mice (FIG. 15D).

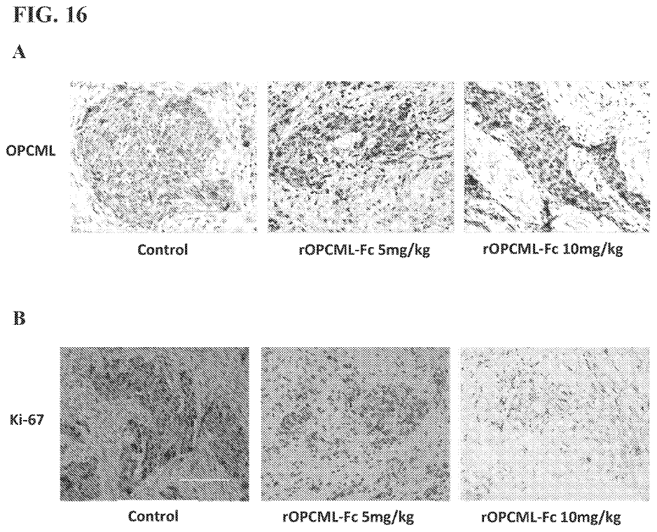

Immunohistochemistry was used to identify the cells targeted by rOPCML-Fc (FIG. 16). Using wild-type OPCML and Ki-67 as markers, rOPCML-Fc was found to target tumor cells in the PDX model tumor environment, without aggregation in normal mouse organs, and inhibited the proliferation of cancer cells in that model.

The cancer to be treated can be ovarian cancer, breast cancer, renal cancer, gastrointestinal cancer, gastric cancer, colon cancer, liver cancer, cervical cancer, prostate cancer, endometrial, endometrial serous carcinoma, esophageal, glioma of any type, lung cancer, non-Hodgkin lymphoma, Hodgkin lymphoma, nasopharyngeal, and nasal NK/T lymphoma.

Administration of the rOPCML-Fc protein to patients in need thereof can be most easily accomplished via intravenous injection, however other types of administration are also possible, such as intraperitoneal injection, intrapleural injection, subcutaneous injection, intraventricular injection and intramuscular injection. Additionally it may be inhaled/nebulised or topically applied to the skin and also interventionally injected intra-arterially to blood supply of tumor deposits.

In some aspects, pharmaceutical compositions comprising the rOPCML-Fc protein and a pharmaceutically acceptable carrier are useful. Here, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active rOPCML-Fc ingredient, its use in the therapeutic or pharmaceutical compositions is contemplated.

Pharmaceutical compositions can be prepared according to conventional mixing, granulating or coating methods and the present pharmaceutical compositions can contain from about 0.1% to about 99%, from about 5% to about 90%, or from about 1% to about 20% of the rOPCML-Fc protein by weight or volume.

In one aspect, rOPCML-Fc is administered in therapeutically effective amounts in a combination therapy with one or more therapeutic agents (pharmaceutical combinations) or modalities, e.g. non-drug therapies. For example, synergistic effects can occur with other anti-proliferative, anti-cancer, immunomodulatory or anti-inflammatory substances. In particular, receptor tyrosine kinase inhibitors may be co administered with rOPCML-Fc. Where the compounds of the invention are administered in conjunction with other therapies, dosages of the co-administered compounds will vary depending on the type of co-drug employed, on the specific drug employed, on the condition being treated, and so forth.

Combination therapy includes administration of a compound of the invention in combination with one or more other biologically active ingredient(s), including but not limited to, a second and different antineoplastic agent or a second agent that targets DNA repair and non-drug therapies, including but not limited to, surgery or radiation treatment. For instance, rOPCML-Fc protein can be used in combination with other pharmaceutically active compounds, preferably biologics or compounds that are able to enhance the effect of the rOPCML-Fc protein. The rOPCML-Fc protein can be administered simultaneously or sequentially to the other drug therapy or treatment modality, e.g. as a single preparation or a separate preparation. In general, a combination therapy envisions administration of two or more biologics or drugs during a single cycle or course of therapy.

In one aspect, enhancing the chemotherapeutic treatment of cancer in a mammal undergoing treatment with an anti-cancer agent is accomplished by co-administering to the mammal an effective amount of rOPCML-Fc protein. In certain aspects, the anti-cancer agent is a DNA damaging agent. The DNA damaging agent can be any suitable DNA damaging agent. Non-limiting examples of suitable DNA damaging agents include DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, tenyposide, triethylenethiophosphoramide and etoposide. The DNA damaging agent can also be radiation or a biotherapeutic agent such as antibody.

In one aspect, the therapeutically effective dose is from about 0.01 mg to about 5,000 mg per day of an rOPCML-Fc protein. The rOPCML-Fc protein or rOPCML-Fc pharmaceutical compositions therefore should provide a dosage of from about 0.01 mg to about 5000 mg of rOPCML-Fc. In certain aspects, pharmaceutical dosage unit forms are prepared to provide from about 1 mg to about 2000 mg, from about 10 mg to about 1000 mg, from about 20 mg to about 500 mg or from about 25 mg to about 250 mg of the essential rOPCML-Fc active ingredient or a combination of essential ingredients per dosage unit form. In certain embodiments, the pharmaceutical dosage unit forms are prepared to provide about 10 mg. 20 mg. 25 mg, 50 mg, 100 mg. 250 mg, 500 mg, 1000 mg or 2000 mg of the essential rOPCML-Fc active ingredient.

The following Examples are just intended to further illustrate certain aspects of the disclosure and are by no means intended to limit the scope.

### EXAMPLES

### Example 1—r-OPCML Signal Peptide Efficiency Determination

Constructs for recombinant OPCML (r-OPCML) molecules were prepared by sending sequences to Invitrogen for generation of expression vectors. To ensure the passage of the anchorless recombinant OPCML through the plasma membrane of HEK-293 cells and into the cytoplasm, all constructs eliminated the last 30 amino acids of the wildtype OPCML protein sequence which contained the GPI-anchor. Briefly the encoded amino acid sequence for the desired signal peptide, all three OPCML Ig domains (D1, D2, and D3, OPCML residues 36-316), the human rhinovirus 3C protease cleavage site (LEVLFQGP; SEQ ID NO: 11), and a His tag was placed into a PCDNA3.1™ expression vector (THERMOFISHER®). The amino acid sequence for the native OPCML signal peptide sequence, the native linker sequence and the first 10 amino acids of the OPCML protein (SEQ ID NO: 3), the ceruloplasmin signal peptide sequence and the first 10 amino acids of the OPCML protein (SEQ ID NO: 4), and the Human Serum Albumin (HSA) signal peptide and the first 10 amino acids of the OPCML protein (SEQ ID NO: 5) are presented in FIG. 2.

Upon receipt of the expression vector constructs, HEK-293 cells were cultured in DMEM high-glycemic culture medium containing 10% fetal bovine serum, 1% penicillin and 1% streptomycin at 37° C. and 5% CO₂. Transfection was accomplished according to the manufacturer's directions using the EFFECTENE® transfection reagent system (QIAGEN®) and 1 μg/μl of pcDNA3.1-rOPCML plasmid.

A single stable transgenic cell line was selected for each construct and cultured in DMEM high-glycemic culture medium containing 10% fetal bovine serum. 1% penicillin and 1% streptomycin at 37° C. and 5% CO₂. Culture medium supernatant was filtered with a 0.45 μm organic filter membrane prior to purification via Ni-NTA resin (QIAGEN®) according to manufacturer's directions.

Purified protein was soluble. The extent to which the signal peptide sequence was able to transfer the expressed recombinant OPCML protein into the media was determined by separation of purified protein on an 8% SDS-PAGE gel, transferred to a nitrocellulose or polyvinylidene fluoride (PVDF) membrane (Millipore Corporation), blocked, and incubated with polyclonal goat anti-human OBCAM (OPCML; R&D Systems), after which densitometry was conducted with a Sapphire FL bimolecular imager for western densitometry and Image J software (Azure Biosystems). FIGS. 2B and 2C show the results. Protein expression from the construct having the native OPCML signal peptide was significantly lower than that found for the ceruloplasmin signal peptide construct and the HSA signal peptide construct (PYTX-001). Consequently, since the HSA signal peptide showed the strongest activity, the PYTX-001 construct was used for subsequent experimentation. The entire amino acid sequence (SEQ ID NO: 6) for the PYTX-001 protein is shown in FIG. 3C.

### Example 2—rOPCML-FC Molecule PYTX-004

A construct for a recombinant OPCML (r-OPCML) molecule having a human IgG Fc fragment containing only the CH2CH3 region (accession 3AGV_A; SEQ ID NO: 12) was prepared by sending the desired amino acid sequence (SEQ ID NO: 10) to Invitrogen for generation of an expression vector. Here, the amino acid sequence of the human rhinovirus 3C protease cleavage site (LEVLFQGP; SEQ ID NO: 11) was inserted along with a linker and a wildtype human IgG Fc region (SEQ ID NO: 13), which contains only the CH2CH3 region of human IgG Fc and lacks the 23 amino acid hinge region which contains sites for disulfide pairing. This prevented Fc dimerization and its potential to interfere with the dimerization of the OPCML D1 domains. In addition, a His-tag was inserted at the C-terminal of the Fc sequence. This resulted in the rOPCML-Fc construct (PYTX-004; FIG. 4).

Upon receipt of the construct, HEK-293 cells were cultured in DMEM high-glycemic culture medium containing 10% fetal bovine serum, 1% penicillin and 1% streptomycin at 37° C. and 5% CO₂. Transfection was accomplished according to the manufacturer's directions using the EFFECTENE® transfection reagent system (QIAGEN®) and 1 μg/μl of pcDNA3.1-rOPCML-Fc plasmid. Control did not contain the pPCDNA3.1-rOPCML-Fc plasmid (PYTX-004 construct). Cells were cultured in DMEM high-glycemic culture medium containing 10% fetal bovine serum, 1% penicillin and 1% streptomycin at 37° C. and 5% CO₂. After 48 hours, media was changed and fresh media containing 800 μg/ml G418 added. Further culturing was done with the G418 media.

Fourteen days after transfection, the cells transfected with rOPCML-Fc showed colony growth while the control did not (FIG. 5A). Individual cells were isolated, grown in culture medium for three days, and the relative content of rOPCML-Fc protein in the supernatant of each clone's culture medium was detected using an ELISA assay (FIG. 5B). The clone with the highest rOPCML-Fc protein content in its medium supernatant, 18G9, was used for all further experiments.

Since the serum in cell culture media affects subsequent protein purification, a serum-free domestication culture was implemented to gradually reduce the cells' dependence on serum. After cells reached 100% confluence, the amount of serum in the culture medium was reduced, which resulted in a decrease in confluence to about 80% for medium with a 5% concentration of serum and a decrease in confluence to about 50% for serum-free medium (FIG. 5C).

The rOPCML-Fc clone was cultured and 1 L of culture medium supernatant was filtered with a 0.45 μm organic filter membrane prior to purification via Ni-NTA resin (QIAGEN®) according to manufacturer's directions. Briefly, cell culture media was applied to the column and eluted with 200 mM imidazole elution buffer. Samples of the cell culture medium before purification, the effluent after purification, the washing solution, and the eluate from different tubes were collected for SDS-PAGE detection. The following samples were tested: cell culture medium pre-binding, effluent, wash solution, and six separate samples of purified rOPCML-Fc protein collected/eluted sequentially.

Samples were separated on an 8% SDS-PAGE gel, transferred to a nitrocellulose or polyvinylidene fluoride (PVDF) membrane (Millipore Corporation), blocked, and incubated with polyclonal goat anti-human OBCAM (OPCML; R&D Systems) as a primary antibody, diluted 1:1000, at 4° C. overnight. After washing, the membrane was incubated with HRP-labeled anti-goat as the secondary antibody, diluted 1:5000. After final washings, proteins were detected using Immobilon Western Chemiluminescent HRP substrate system (Millipore) and GE Healthcare Amersham “Hyperfilm” ECL film with a Kodak SRX2000 (Rochester, NY, USA) developer machine. Results are shown in FIG. 5D. Here, more than 95% of the rOPCML-Fc proteins were still exhibiting two forms after purification; one in the form of a polymer with a molecular weight of 160 kDa, and one in the form of a monomer with 80 kDa. The lack of signal for the effluent after purification indicated that the binding efficiency reached 100%.

Purified protein was soluble and expression of the rOPCML-Fc protein determined by Western blotting. Here, purified protein sample was separated on an 8% SDS-PAGE gel, transferred to a nitrocellulose or polyvinylidene fluoride (PVDF) membrane (Millipore Corporation), blocked, and incubated with polyclonal goat anti-human OBCAM (OPCML; R&D Systems) as a primary antibody, diluted 1:1000, at 4° C. overnight. After washing, the membrane was incubated with HRP-labeled anti-goat as the secondary antibody, diluted 1:5000. After final washings, proteins were detected using Immobilon Western Chemiluminescent HRP substrate system (Millipore) and GE Healthcare Amersham “Hyperfilm” ECL film with a Kodak SRX2000 (Rochester, NY, USA) developer machine. HPLC was conducted on a Perkin Elmer LC 300 HPLC machine according to manufacturer's directions. Results are shown in FIG. 7A and FIG. 7B.

### Example 3—Assessing rOPCML-Fc (PYTX-004 Construct) Signaling Potency

To confirm that the Fc portion of rOPCML-Fc did not adversely affect its signaling ability/potency. Westerns were conducted comparing the activity of rOPCML (PYTX-001 construct) with rOPCML-Fc (PYTX-004 construct) Here, SKOV3 and OVCAR8 cells in the logarithmic growth phase were used. Serum starvation and synchronization of the cells was achieved by culturing in medium without fetal bovine serum for 24 hours. Medium was replaced with medium containing 10% fetal calf serum (FCS) and 40 μg/ml of rOPCML (PYTX-001 construct) protein or 40 μg/ml of rOPCML-Fc (PYTX-004 construct) protein. Controls consisted of the addition of medium containing only 10% FCS or the addition of unaltered medium. Protein was extracted after 30 minutes of time.

Protein samples were separated on an 8% SDS-PAGE gel and transferred to a nitrocellulose or PVDF membrane, blocked, and incubated with primary antibody, diluted 1:1000, at 4° C. overnight. Primary antibodies used were: phosph-HER2/ErbB2 (T1248) (rabbit, Cell signaling Technology); Total-HER2 (rabbit, Cell signaling Technology); human phospho-Axl (Y779) (rabbit, R&D Systems); phospho-AKT (S473) (rabbit, Cell Signaling Technology); Pan-AKT (rabbit, Cell Signaling Technology); phospho ERK1/2 (T202/Y204) (rabbit. Cell Signaling Technology); Total-ERK (rabbit, Cell Signaling Technology); Calnexin (rabbit. Cell Signaling Technology); OPCML (goat, R&D Systems).

After washing, the membrane was incubated with HRP-labeled secondary antibodies (i.e. anti-rabbit or anti-goat), diluted 1:5000. After final washings, proteins were detected using Immobilon Western Chemiluminescent HRP substrate system (Millipore) and GE Healthcare Amersham “Hyperfilm” ECL film with a Kodak SRX2000 (Rochester, NY, USA) developer machine. Results are presented in FIG. 6 and indicate similarities between rOPCML and rOPCML-Fc protein for phospho-AXL-Y779, phospho-AKT-S473, and phospho-ERK-1/2-T202-Y204. Both the r-OPCML protein (the PYTX-001 construct) and the rOPCML-Fc protein (the PYTX-004 construct) appeared essentially equipotent for on-target and downstream pharmacodynamics.

### Example 4—IC50 Test of rOPCML-Fc Fusion Protein (PYTX-004)

To determine the IC50 value for the rOPCM-Fc fusion protein, three different cell lines were selected: OVCAR3, SKOV3, and PEO1. Briefly, cells in the logarithmic growth stage were washed with PBS buffer, treated for 1-2 minutes with 0.25% Trypsin-EDTA at 37° C. and 5% CO₂, and then treated with medium containing 10% fetal bovine serum. Cells were isolated, diluted, further sub-cultured at 37° C. and 5% CO₂, and treated with culture medium containing 10% fetal bovine serum, penicillin, and streptomycin to which was added the following concentrations of rOPCML-Fc protein: 0.0 mg/ml, 0.025 mg/ml, 0.05 mg/ml, 0.1 mg/ml, 0.2 mg/ml, and 0.8 mg/ml. Cells were cultured for 6 day in a 96-well plate, then each well treated with 100 μl medium containing 10% CCK8 reagent, and incubated at 37° C. with 5% CO₂ for 1.5-2 hours prior to taking an absorbance reading at A450 nm. Results are presented in FIG. 8A, showing an IC50 of 0.379 for OVCAR-3 cells, 0.519 for SKOV3 cells, and 0.612 for PEO1.

The ability of different concentrations of rOPCML-Fc protein (PYTX-004 construct) to inhibit cell OVCAR3 and SKOV3 cell growth was also determined. Here, cells were prepared as above. Concentrations of 0.8 mg/ml, 0.4 mg/ml, 0.2 mg/mg, 0.1 mg/ml, 0.05 mg/ml, 0.025 mg/ml, and 0.0 mg/ml of rOPCML-Fc protein were added to the cell cultures for time periods of 48, 96, and 144 hours (FIG. 8B).

### Example 5—Effect of rOPCML-Fc Protein (PYTX-004 Construct) on Wound Healing

The in vitro gap closure assay set forth in Birtley et al (2019. Nature Communications 10:3134) was performed using OVCAR3 and SKOV3cells to determine the effect of rOPCML-Fc protein (PYTX-004 construct) on wound healing. Briefly, cell lines were seeded at 100% confluence into culture inserts in a 24-well plate and serum starved overnight. The insert was removed to generate a 500 μm gap, and cells stimulated with 0.05 mg/ml or 0.1 mg/ml of rOPCML-Fc protein. Control cells were not stimulated. After 24 hours, cells were visualized under the microscope (FIGS. 9A and 9B).

Both concentrations inhibited the ability of OVCAR3 and SKOV3 cells to infiltrate and close a gap within 24 hours of rOPCML-Fc protein application. FIGS. 9C and 9D show the results in graph form. Notably, the administration of 0.1 mg/ml rOPCML-Fc was highly significant for both cell types as compared to the control.

### Example 6—Effect of rOPCML-Fc Protein (PYTX-004 Construct) on Invasion Ability of OVCAR3 and SKOV3 Cells

The Birtley et al. (2019, Nature Communications 10:3134) invasion assay was used to assess the ability of rOPCML-Fc to inhibit OVCAR3 and SKOV3 cells. Here, fertilized chicken eggs were prepared as set forth in Birtley et al. On ED9, grafts were prepared by suspending 106 cells of either OVCAR3 and SKOV3 cells in 100 μl of Matrigel (BD Biosciences). The following concentrations of rOPCML-Fc protein was added to the grafts before being deposited onto the membrane and re-covering and sealing the window: 0.0 mg/ml. 0.05 mg/ml, or 0.1 mg/ml.

The results shown in FIG. 10A-C show that rOPCML-Fc protein had positive inhibitory effects at both concentrations. However, the difference between the control and administration of 0.1 mg/ml rOPCML-Fc protein was highly significant.

### Example 7—Effect of rOPCML-Fc Protein (PYTX-004 Construct) on Ovarian Cancer Patient-Derived Organoids (PDO)

To further characterize the inhibitory potential of rOPCML-Fc, experiments were conducted with ovarian cancer patient-derived organoids (PDO). Here, patient-derived organoids of patients with ovarian cancer were derived from the PDO tissue sample library constructed by Beijing Keyu Technology Co., Ltd., numbered OV016X-PDO, OV009-PDO, OV044-PDO, OV041-PDO and OV024-PDO. All of the organoids were derived from ovarian tumor tissues obtained during the initial operation of patients who were pathologically diagnosed as high-grade serous carcinoma of the ovary and were not treated.

Briefly, organoid tissues were collected and digested for 15 minutes with TRYPLE™ digestion solution containing 10 μM Y-27632 with shaking at 37° C. The digestion was stopped by addition of Advanced-DMEM/F12 medium containing 5% fetal bovine serum (FBS) prior to centrifugation at 1000 rpm for 5 minutes. Cells were resuspended in GAS-Ad-ES medium, diluted to 8×104 cells/ml prior to mixing with Matrigel at a ratio of 1:1, and 0.5 ml added to each well in a 24-well plate. Cells were incubated at 37° C., 5% constant temperature incubator for 30 to solidify prior to the addition of GAS-Ad-ES medium and continued incubation. Fresh medium was added every 3 days. Experiments were carried out after 3-4 passages.

For determining the effect of rOPCML-Fc on PDO, 1.6×105 cells/ml in GAS-Ad-ES medium were mixed with Matrigel at a ratio of 1:1 and 50 μl/well added to a low-adhesion 96-well plate. Cells were incubated at 37° C., 5% constant temperature incubator for 30 to solidify prior to the addition of 40 μl GAS-Ad-ES medium per well and continued incubation at 37° C. for 2 days. Cells were then divided into 6 concentration groups, each with 3 repeating wells and each well supplemented with 30 μl of GAS-Ad-Es medium containing either 0.05, 0.1, 0.2, 0.4, or 0.8 mg/ml rOPCML-Fc. Control wells were supplemented with 60 μl of GAS-Ad-Es medium. Plates were then incubated at 37° C., 5% constant temperature incubator for 2 days, after which wells were replenished with 30 μl of the appropriate rOPCML-Fc mixture or with GAS-Ad-Es medium for control wells. Cells were further cultured for 2 days at 37° C., 5% constant temperature incubator.

Chemiluminescence was then measured to evaluate cell viability. Here. 10 ml CELLTITER_GLO® buffer was added to 1 vial of CELLTITER_GLO® buffer substrate and mixed well. 50 μl of the resulting CELLTITER_GLO® mixture was added to each well and the microplate gently mixed for 2 minutes on a shaker. The plate was incubated for 10 minutes in the dark at room temperature prior to placing on a multifunctional microplate reader to detect luminescence value. Results are shown in FIG. 11A and FIG. 11B, indicating a statistically significant decrease in cell viability/proliferation activity for administration of 0.4 and 0.8 mg/ml rOPCML-Fc in OV016X PDO, a statistically significant decrease in viability/proliferation activity for administration of 0.8 mg/ml rOPCML-Fc in OV044 PDO, and a highly statistically significant decrease in viability/proliferation activity for administration of 0.8 mg/ml rOPCML-Fc in OV041 PDO.

### Example 8—rOPCML-Fc Effect on Signaling Pathways

The signaling ability of rOPCML-Fc was investigated and the results presented in FIG. 12. Here, OVCAR3 cells in the logarithmic growth phase were used. Serum starvation and synchronization of the cells was achieved by culturing in medium without fetal bovine serum for 24 hours. Medium was replaced with medium containing 50 ng/ml of EGF and 0.1 mg/ml of rOPCML-Fc (PYTX-004 construct) protein. Controls consisted of the addition of only medium containing 50 ng/ml of EGF or the addition of unaltered medium. Protein was extracted after 30 minutes of time.

Protein samples were separated on an 8% SDS-PAGE gel and transferred to a nitrocellulose or PVDF membrane, blocked, and incubated with primary antibody, diluted 1:1000, at 4° C. overnight. Primary antibodies used were: phosph-HER2/ErbB2 (T1248) (rabbit, Cell Signaling Technology); Total-HER2 (rabbit, Cell Signaling Technology); human phospho-Axl (Y779) (rabbit, R&D Systems); phospho-AKT (S473) (rabbit, Cell Signaling Technology); p-AKT (rabbit, Cell Signaling Technology), GAPDH (rabbit, Cell Signaling Technology), p-EGFR (rabbit, Cell Signaling Technology), p-RAF (rabbit. Cell Signaling Technology); p-MEK (rabbit, Cell Signaling Technology); p-ERK (rabbit, Abcam).

After washing, the membrane was incubated with HRP-labeled secondary antibodies (i.e. anti-rabbit), diluted 1:5000. After final washings, proteins were detected using Immobilon Western Chemiluminescent HRP substrate system (Millipore) and GE Healthcare Amersham “Hyperfilm” ECL film with a Kodak SRX2000 (Rochester, NY, USA) developer machine. Results are presented in FIG. 12. rOPCML-Fc downregulated pHER2, and the RAF/MEK/ERK pathway in ovarian cancer cells.

### Example 9—Construction of PDX Model of NDG Mouse Ovarian Cancer

A flowchart for the generation of the PDX model is shown in FIG. 13. Here, the omentum metastasis from a patient with high-grade serous carcinoma of the ovary was inoculated subcutaneously on the backs of 7 NDG mice, and each NDG mouse was inoculated at 3 sites on the back. The transplanted tumor growth was seen on the back of 7 mice, a total of 13/21. Successful modeling of each site, this is the P1 generation transplantation tumor, the success rate is 61.9%. A total of 12 NDG mice were inoculated with the transplanted tumors of the P2 generation. Three mice died of illness one week after the inoculation. The transplanted tumors on the backs of the remaining 9 mice all grew well, and the transplantation success rate was 75%. After tumor formation, the tumor tissue of NDG mice was cut and observed. The transplanted tumor was round or nodular, with solid flesh on the cut surface and abundant capillaries on the surface.

The original tumor tissue, P1, and P2 generation transplanted tumors were stained with HE and observed under a light microscope (FIG. 14). The histological morphology of P1 and P2 generations was highly similar to the original tumor tissue morphology and the cell arrangement There was no polarity, large nucleus, and deep staining. Compared with the original tumor tissue morphology, the P1 and P2 generation transplanted tumors were less differentiated and more malignant. To identify whether the transplanted tumor tissue retained the molecular phenotypic characteristics of the original tumor tissue, PAX-8 immunohistochemical staining was performed on the original tumor tissue, P1 generation, and P2 generation transplanted tumors. The original tumor tissue, P1 generation, and P2 generation. The PAX-8 of the next-generation xenograft tumor specimens were all positively expressed.

### Example 10—Intravenous Application of rOPCML-Fc (PYTX-004 Construct)

P2 generation mice were used. When transplanted tumor volume reached 100 mm³, animals were divided into 3 groups. The experimental group was given 5 mg/kg and 10 mg/kg rOPCML-Fc fusion protein according to the body weight of NDG mice, and the control group received the same volume of phosphate buffered saline (PBS); all were injected into the tail vein every other day. The long and short diameters of the tumor volume on the back of NDG mice were measured every 4 days. Animals were sacrificed at 28 days of treatment, and the growth curve of the transplanted tumor plotted. When the volume of the tumors before treatment was basically the same treatment commenced; the tumor volume in the control group showed progressive increase, while the volume of the two rOPCML-Fc fusion protein treatment groups progressively decreased. A clear dose-dependent statistically significant reduction in the tumor volume in the treated animals (p=0.010, p=0.002) was evident.

The transplanted tumors of each group of animals were stripped and weighed (FIG. 15A). The results showed that compared with the average tumor weight of the control group of 79.87±51.81 mg, the average tumor weight of the 5 mg/kg rOPCML-Fc fusion protein group was 18.13±8.64 mg, the inhibition rate was 77.3%; the average tumor weight of the 10 mg/kg rOPCML-Fc fusion protein group was 5.53±1.26 mg, and the tumor weight reduction rate was 93.0% (FIG. 15B-D).

### Example 11—Immunohistochemical Analysis of rOPCML-Fc Protein after Treatment of P2 Generation Transplanted Tumor

Ki-67 is a related antigen of proliferating cells, which is used to mark cells in the proliferation cycle. The cells that are positive for this mark grow faster and have poor tissue differentiation ability. An immunohistochemical method was used to detect the expression of Ki-67 and the binding of rOPCML-Fc to P2 generation transplanted tumors after administration of rOPCML-Fc protein.

Results showed Ki-67 nuclear staining. Compared with the control group, Ki-67 expression was significantly reduced in the 5 mg/kg and 10 mg/kg rOPCML-Fc protein treatment groups (FIG. 16B), indicating that its proliferation activity was inhibited.

At the same time, immunohistochemistry to detect the binding activity of rOPCML-Fc protein to transplanted tumors. Results showed in the 5 mg/kg and 10 mg/kg rOPCML-Fc protein treatment groups, rOPCML-Fc specifically targets cancer cells in transplanted tumors, but the content of mesenchymal cells is low (FIG. 16A).

1. A polypeptide molecule comprising an amino acid sequence having 95% identity to SEQ ID NO: 6.

2. The polypeptide molecule according to claim 1, wherein the sequence identity is 100%.

3. A pharmaceutical composition comprising the polypeptide molecule according to claim 1 and a pharmaceutically acceptable carrier.

4. The pharmaceutical composition according to claim 3, wherein the pharmaceutically acceptable carrier is suitable for intravenous administration.

5. A nucleic acid molecule comprising a nucleotide sequence encoding an amino acid sequence having at least 95% identity to SEQ ID NO: 6.

6. The nucleic acid molecule according to claim 5, wherein the sequence identify is 100%.

7. A vector construct comprising:
(a) A first nucleic acid having a regulatory sequence that causes transcription and/or translation; and
(b) A second nucleic acid having the nucleotide sequence according to claim 5,
wherein the first and second nucleic acids are operably linked.

8. A host cell comprising a nucleic acid molecule according to claim 5.

9. A host cell comprising a vector construct according to claim 7.

10. A method of introducing a nucleic acid into a host cell comprising:
(a) providing a nucleic acid molecule according to claim 5; and
(b) contacting the nucleic acid with the host cell under conditions that permit insertion of the nucleic acid into the host cell.

11. A method of transfecting a host cell that comprises contacting a host cell with a vector construct according to claim 7.

12. A method of treating a disease or condition comprising administering to a patient in need thereof an effective amount of the polypeptide molecule according to claim 1.

13. The method according to claim 12, wherein the disease or condition is HER2-postive cancer or is HER-negative cancer.

14. (canceled)

15. The method according to claim 12, wherein the disease or condition is cancer selected from the group consisting of ovarian cancer, breast cancer, renal cancer, gastrointestinal cancer, gastric cancer, colon cancer, liver cancer, cervical cancer, prostate cancer, endometrial, endometrial serous carcinoma, esophageal, glioma of any type, lung cancer, non-Hodgkin lymphoma, Hodgkin lymphoma, nasopharyngeal, and nasal NK/T lymphoma.

16. The method according to claim 15, wherein the cancer is ovarian cancer.

17. The method according to claim 12, wherein the polypeptide molecule is administered intravenously.

18. A method of treating a disease or condition comprising administering to a patient in need thereof an effective amount of the pharmaceutical composition according to claim 3.

19. The method according to claim 18, wherein the disease or condition is HER2-postive cancer or is HER-negative cancer.

20. (canceled)

21. The method according to claim 18, wherein the disease or condition is cancer selected from the group consisting of ovarian cancer, breast cancer, renal cancer, gastrointestinal cancer, gastric cancer, colon cancer, liver cancer, cervical cancer, prostate cancer, endometrial, endometrial serous carcinoma, esophageal, glioma of any type, lung cancer, non-Hodgkin lymphoma, Hodgkin lymphoma, nasopharyngeal, and nasal NK/T lymphoma.

22. The method according to claim 21, wherein the cancer is ovarian cancer.

23. The method according to claim 18, wherein the polypeptide molecule is administered intravenously.


SEQUENCE LISTING (14 sequences)

SEQ ID NO: 1 - protein, 345 aa, Homo sapiens
  REGION 1..345: note = misc_feature - Wildtype GPI Anchored human OPCML
MGVCGYLFLPWKCLVVVSLRLLFLVPTGVPVRSGDATFPKAMDNVTVRQGESATLRCTIDDRVTRVAWLNRSTILYAGNDKWSIDPRVIILVNTPTQYSIMIQNVDVYDEGPYTCSVQTDNHPKTSRVHLIVQVPPQIMNISSDITVNEGSSVTLLCLAIGRPEPTVTWRHLSVKEGQGFVSEDEYLEISDIKRDQSGEYECSALNDVAAPDVRKVKITVNYPPYISKAKNTGVSVGQKGILSCEASAVPMAEFQWFKEETRLATGLDGMRIENKGRMSTLTFFNVSEKDYGNYTCVATNKLGNTNASITLYGPGAVIDGVNSASRALACLWLSGTLLAHFFIKF

SEQ ID NO: 2 - protein, 27 aa, Homo sapiens
  REGION 1..27: note = misc_feature - Wildtype human OPCML signal peptide sequence
MGVCGYLFLPWKCLVVVSLRLLFLVPT

SEQ ID NO: 3 - protein, 45 aa, Homo sapiens
  REGION 1..45: note = misc_feature - Wildtype human OPCML signal peptide sequence, wildtype linker and first 10 amino acids of the human OPCML protein
MGVCGYLFLPWKCLVVVSLRLLFLVPTGVPVRSGDATFPKAMDNV

SEQ ID NO: 4 - protein, 28 aa, Homo sapiens
  REGION 1..28: note = misc_feature - Human Ceruloplasmin signal peptide sequence and first 10 amino acids of the human OPCML protein
MKILILGIFLFLCSTPLQATFPKAMDNV

SEQ ID NO: 5 - protein, 28 aa, Homo sapiens
  REGION 1..28: note = misc_feature - Human Serum Albumin signal peptide sequence and first 10 amino acids of the human OPCML protein
MKWVTFISLLFLFSSAYSATFPKAMDNV

SEQ ID NO: 6 - protein, 334 aa, synthetic construct
  REGION 1..334: note = Synthetic: Sequence for PYTX-001 construct
MKWVTFISLLFLFSSAYSGVPVRSGDATFPKAMDNVTVRQGESATLRCTIDDRVTRVAWLNRSTILYAGNDKWSIDPRVIILVNTPTQYSIMIQNVDVYDEGPYTCSVQTDNHPKTSRVHLIVQVPPQIMNISSDITVNEGSSVTLLCLAIGRPEPTVTWRHLSVKEGQGFVSEDEYLEISDIKRDQSGEYECSALDVAAPDVRKVKITVNYPPYISKAKNTGVSVGQKGILSCEASAVPMAEFQWFKEETRLATGLDGMRIENKGRMSTLTFFNVSEKDYGNYTCVATNKLGNTNASITLYGPGGGGLEVLFQGPRTGHHHHHHSSLEGPRFE

SEQ ID NO: 7 - protein, 18 aa, Homo sapiens
  REGION 1..18: note = misc_feature - Human serum albumin signal peptide sequence
MKWVTFISLLFLFSSAYS

SEQ ID NO: 8 - protein, 47 aa, Homo sapiens
  REGION 1..47: note = misc_feature - Wildtype OPCML sequence surrounding and including the GPI anchor site
TNKLGNTNASITLYGPGAVIDGVNSASRALACLWLSGTLLAHFFIKF

SEQ ID NO: 9 - protein, 46 aa, synthetic construct
  REGION 1..46: note = Synthetic: His tagged sequence
TNKLGNTNASITLYGPGGGGLEVLFQGPRTGHHHHHHSSLEGPRFE

SEQ ID NO: 10 - protein, 534 aa, synthetic construct
  REGION 1..534: note = Sequence for construct PYTX-004
MKWVTFISLLFLFSSAYSATFPKAMDNVTVRQGESATLRCTIDDRVTRVAWLNRSTILYAGNDKWSIDPRVIILVNTPTQYSIMIQNVDVYDEGPYTCSVQTDNHPKTSRVHLIVQVPPQIMNISSDITVNEGSSVTLLCLAIGRPEPTVTWRHLSVKEGQGFVSEDEYLEISDIKRDQSGEYECSALNDVAAPDVRKVKITVNYPPYISKAKNTGVSVGQKGILSCEASAVPMAEFQWFKEETRLATGLDGMRIENKGRMSTLTFFNVSEKDYGNYTCVATNKLGNTNASITLYGPGGGGLEVLFQGPGGSTKGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTGHHHHHH

SEQ ID NO: 11 - protein, 8 aa, unidentified
  REGION 1..8: note = Human rhinovirus 3C protease cleavage site
LEVLFQGP

SEQ ID NO: 12 - protein, 211 aa, Homo sapiens
  REGION 1..211: note = misc_feature - Human IgG1 CH2CH3 Fc fragment (accession 3AGV_A)
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 216 aa, synthetic construct
  REGION 1..216: note = Synthetic: Engineered linker and human IgG1 CH2Ch3 Fc fragment
GGSTKGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 18 aa, Homo sapiens
  REGION 1..18: note = misc_feature - Human Ceruloplasmin signal peptide sequence
MKILILGIFLFLCSTPLQ